(19) United States
(12) Patent Application Publication
(10) Pub. No.: US 2003/0148985 A1
(43) Pub. Date: Aug. 7, 2003

(54) Methods and reagents for the inhibition of hepatitis B virus replication

(76) Inventors: David Morrissey, Boulder, CO (US); James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/310,294

(22) Filed: Dec. 5, 2002

Related U.S. Application Data
(60) Provisional application No. 60/337,055, filed on Dec. 5, 2001.

(51) Int. Cl. A61K 48/00; C07H 21/04; A61K 38/21

(52) U.S. Cl. 514/44; 424/85.5; 536/23.72

(57) ABSTRACT

The present invention relates to nucleic acid molecules that modulate Hepatitis B virus (HBV) gene expression and HBV replication, and methods thereof. Specifically, the present invention relates to nucleic acid decoy molecules and aptamers that bind to HBV reverse transcriptase and/or HBV reverse transcriptase primer sequences and methods for their use alone or in combination with other therapies. The present invention also relates to nucleic acid molecules that specifically bind the Enhancer I region of HBV DNA and methods for their use.

### BACKGROUND OF THE INVENTION

The present invention concerns compounds, compositions, and methods for the study, diagnosis, and treatment of degenerative and disease states related to hepatitis B virus (HBV) infection, replication and gene expression. Specifically, the invention relates to nucleic acid molecules used to modulate expression of HBV.

The following is a discussion of studies relating to hepatitis B virus (HBV). The discussion is not meant to be complete and is provided only for understanding of the invention that follows. The summary is not an admission that any of the work described below is prior art to the claimed invention.

Chronic hepatitis B is caused by an enveloped virus, commonly known as the hepatitis B virus or HBV. HBV is transmitted via infected blood or other body fluids, especially saliva and semen, during delivery, sexual activity, or sharing of needles contaminated by infected blood. Individuals may be “carriers” and transmit the infection to others without ever having experienced symptoms of the disease. Persons at highest risk are those with multiple sex partners, those with a history of sexually transmitted diseases, parenteral drug users, infants born to infected mothers, “close” contacts or sexual partners of infected persons, and healthcare personnel or other service employees who have contact with blood. Transmission is also possible via tattooing, ear or body piercing, and acupuncture; the virus is also stable on razors, toothbrushes, baby bottles, eating utensils, and some hospital equipment such as respirators, scopes and instruments. There is no evidence that HBsAg positive food handlers pose a health risk in an occupational setting, nor should they be excluded from work. Hepatitis B has never been documented as being a food-borne disease. The average incubation period is 60 to 90 days, with a range of 45 to 180; the number of days appears to be related to the amount of virus to which the person was exposed. However, determining the length of incubation is difficult, since onset of symptoms is insidious. Approximately 50% of patients develop symptoms of acute hepatitis that last from 1 to 4 weeks. Two percent or less of these individuals develop fulminant hepatitis resulting in liver failure and death.

The determinants of severity include: (1) The size of the dose to which the person was exposed; (2) the person's age with younger patients experiencing a milder form of the disease; (3) the status of the immune system with those who are immunosuppressed experiencing milder cases; and (4) the presence or absence of co-infection with the Delta virus (hepatitis D), with more severe cases resulting from co-infection. In symptomatic cases, clinical signs include loss of appetite, nausea, vomiting, abdominal pain in the right upper quadrant, arthralgia, and tiredness/loss of energy. Jaundice is not experienced in all cases, however, jaundice is more likely to occur if the infection is due to transfusion or percutaneous serum transfer, and it is accompanied by mild pruritus in some patients. Bilirubin elevations are demonstrated in dark urine and clay-colored stools, and liver enlargement may occur accompanied by right upper-quadrant pain. The acute phase of the disease may be accompanied by severe depression, meningitis, Guillain-Barre syndrome, myelitis, encephalitis, agranulocytosis, and/or thrombocytopenia.

Hepatitis B is generally self-limiting and will resolve in approximately 6 months. Asymptomatic cases can be detected by serologic testing, since the presence of the virus leads to production of large amounts of HBsAg in the blood. This antigen is the first and most useful diagnostic marker for active infections. However, if HBsAg remains positive for 20 weeks or longer, the person is likely to remain positive indefinitely and is now a carrier. While only 10% of persons over age 6 who contract HBV become carriers, 90% of infants infected during the first year of life do so.

Hepatitis B virus (HBV) infects over 300 million people worldwide (Imperial, 1999, Gastroenterol. Hepatol., 14 (suppl), S1-5). In the United States, approximately 1.25 million individuals are chronic carriers of HBV as evidenced by the fact that they have measurable hepatitis B virus surface antigen HBsAg in their blood. The risk of becoming a chronic HBsAg carrier is dependent upon the mode of acquisition of infection as well as the age of the individual at the time of infection. For those individuals with high levels of viral replication, chronic active hepatitis with progression to cirrhosis, liver failure and hepatocellular carcinoma (HCC) is common, and liver transplantation is the only treatment option for patients with end-stage liver disease from HBV.

The natural progression of chronic HBV infection over a 10 to 20 year period leads to cirrhosis in 20-to-50% of patients and progression of HBV infection to hepatocellular carcinoma has been well documented. There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al., 1993, American Journal of Gastroenterology, 88, 240-243). Treatment of hepatocellular carcinoma with chemotherapeutic agents has not proven effective and only 10% of patients will benefit from surgery due to extensive tumor invasion of the liver (Trinchet et al., 1994, Presse Medicine, 23, 831-833). Given the aggressive nature of primary hepatocellular carcinoma, the only viable treatment alternative to surgery is liver transplantation (Pichlmayr et al., 1994, Hepatology., 20, 33S-40S).

Upon progression to cirrhosis, patients with chronic HCV infection present with clinical features, which are common to clinical cirrhosis regardless of the initial cause (D'Amico et al., 1986, Digestive Diseases and Sciences, 31, 468-475). These clinical features may include: bleeding esophageal varices, ascites, jaundice, and encephalopathy (Zakim D, Boyer T D. Hepatology a textbook of liver disease, Second Edition Volume 1. 1990 W. B. Saunders Company. Philadelphia). In the early stages of cirrhosis, patients are classified as compensated, meaning that although liver tissue damage has occurred, the patient's liver is still able to detoxify metabolites in the blood-stream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Hepatitis B virus is a double-stranded circular DNA virus. It is a member of the Hepadnaviridae family. The virus consists of a central core that contains a core antigen (HBcAg) surrounded by an envelope containing a surface protein/surface antigen (HBsAg) and is 42 nm in diameter. It also contains an e antigen (HBeAg), which, along with HBcAg and HBsAg, is helpful in identifying this disease

In HBV virions, the genome is found in an incomplete double-stranded form. HBV uses a reverse transcriptase to transcribe a positive-sense full length RNA version of its genome back into DNA. This reverse transcriptase also contains DNA polymerase activity and thus begins replicating the newly synthesized minus-sense DNA strand. However, it appears that the core protein encapsidates the reverse-transcriptase/polymerase before it completes replication.

From the free-floating form, the virus must first attach itself specifically to a host cell membrane. Viral attachment is one of the crucial steps that determines host and tissue specificity. However, currently there are no in vitro cell-lines that can be infected by HBV. There are some cells lines, such as HepG2, which can support viral replication only upon transient or stable transfection using HBV DNA.

After attachment, fusion of the viral envelope and host membrane must occur to allow the viral core proteins containing the genome and polymerase to enter the cell. Once inside, the genome is translocated to the nucleus where it is repaired and cyclized.

The complete closed circular DNA genome of HBV remains in the nucleus and gives rise to four transcripts. These transcripts initiate at unique sites but share the same 3′-ends. The 3.5-kb pregenomic RNA serves as a template for reverse transcription and also encodes the nucleocapsid protein and polymerase. A subclass of this transcript with a 5′-end extension codes for the precore protein that, after processing, is secreted as HBV e antigen. The 2.4-kb RNA encompasses the pre-S1 open reading frame (ORF) that encodes the large surface protein. The 2.1-kb RNA encompasses the pre-S2 and S ORFs that encode the middle and small surface proteins, respectively. The smallest transcript (˜0.8-kb) codes for the X protein, a transcriptional activator.

Multiplication of the HBV genome begins within the nucleus of an infected cell. RNA polymerase II transcribes the circular HBV DNA into greater-than-full length mRNA. Since the mRNA is longer than the actual complete circular DNA, redundant ends are formed. Once produced, the pregenomic RNA exits the nucleus and enters the cytoplasm.

The packaging of pregenomic RNA into core particles is triggered by the binding of the HBV polymerase to the 5′ epsilon stem-loop. RNA encapsidation is believed to occur as soon as binding occurs. The HBV polymerase also appears to require associated core protein in order to function. The HBV polymerase initiates reverse transcription from the 5′ epsilon stem-loop three to four base pairs at which point the polymerase and attached nascent DNA are transferred to the 3′ copy of the DR1 region. Once there, the (−)DNA is extended by the HBV polymerase while the RNA template is degraded by the HBV polymerase RNAse H activity. When the HBV polymerase reaches the 5′ end, a small stretch of RNA is left undigested by the RNAse H activity. This segment of RNA is comprised of a small sequence just upstream and including the DR1 region. The RNA oligomer is then translocated and annealed to the DR2 region at the 5′ end of the (−)DNA. It is used as a primer for the (+)DNA synthesis which is also generated by the HBV polymerase. It appears that the reverse transcription as well as plus strand synthesis may occur in the completed core particle.

Since the pregenomic RNA is required as a template for DNA synthesis, this RNA is an excellent target for nucleic acid based therapeutics. Nucleoside analogues that have been documented to modulate HBV replication target the reverse transcriptase activity needed to convert the pregenomic RNA into DNA. Nucleic acid decoy and aptamer modulation of HBV reverse transcriptase would be expected to result in a similar modulation of HBV replication.

Therapeutic Approaches

Current therapeutic goals of treatment are three-fold: to eliminate infectivity and transmission of HBV to others, to arrest the progression of liver disease and improve the clinical prognosis, and to prevent the development of hepatocellular carcinoma (HCC).

Interferon alpha use is the most common therapy for HBV; however, recently Lamivudine (3TC®) has been approved by the FDA. Interferon alpha (IFN-alpha) is one treatment for chronic hepatitis B. The standard duration of IFN-alpha therapy is 16 weeks, however, the optimal treatment length is still poorly defined. A complete response (HBV DNA negative HBeAg negative) occurs in approximately 25% of patients. Several factors have been identified that predict a favorable response to therapy including: High ALT, low HBV DNA, being female, and heterosexual orientation.

There is also a risk of reactivation of the hepatitis B virus even after a successful response, this occurs in around 5% of responders and normally occurs within 1 year.

Side effects resulting from treatment with type 1 interferons can be divided into four general categories including: Influenza-like symptoms, neuropsychiatric, laboratory abnormalities, and other miscellaneous side effects. Examples of influenza-like symptoms include, fatigue, fever, myalgia, malaise, appetite loss, tachycardia, rigors, headache and arthralgias. The influenza-like symptoms are usually short-lived and tend to abate after the first four weeks of dosing (Dusheiko et al., 1994, Journal of Viral Hepatitis, 1, 3-5). Neuropsychiatric side effects include irritability, apathy, mood changes, insomnia, cognitive changes, and depression. Laboratory abnormalities include the reduction of myeloid cells, including granulocytes, platelets and to a lesser extent, red blood cells. These changes in blood cell counts rarely lead to any significant clinical sequellae. In addition, increases in triglyceride concentrations and elevations in serum alanine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea, diarrhea, abdominal and back pain, pruritus, alopecia, and rhinorrhea. In general, most side effects will abate after 4 to 8 weeks of therapy (Dushieko et al, supra).

Lamivudine (3TC®) is a nucleoside analogue, which is a very potent and specific inhibitor of HBV DNA synthesis. Lamivudine has recently been approved for the treatment of chronic Hepatitis B. Unlike treatment with interferon, treatment with 3TC® does not eliminate the HBV from the patient. Rather, viral replication is controlled and chronic administration results in improvements in liver histology in over 50% of patients. Phase III studies with 3TC®, showed that treatment for one year was associated with reduced liver inflammation and a delay in scarring of the liver. In addition, patients treated with Lamivudine (100 mg per day) had a 98 percent reduction in hepatitis B DNA and a significantly higher rate of seroconversion, suggesting disease improvements after completion of therapy. However, stopping of therapy resulted in a reactivation of HBV replication in most patients. In addition recent reports have documented 3TC® resistance in approximately 30% of patients.

Current therapies for treating HBV infection, including interferon and nucleoside analogues, are only partially effective. In addition, drug resistance to nucleoside analogues is now emerging, making treatment of chronic Hepatitis B more difficult. Thus, a need exists for effective treatment of this disease that utilizes antiviral modulators that work by mechanisms other than those currently utilized in the treatment of both acute and chronic hepatitis B infections.

Draper, U.S. Pat. No. 6,017,756, describes the use of ribozymes for the inhibition of Hepatitis B Virus.

Passman et al., 2000, Biochem. Biophys. Res. Commun., 268(3), 728-733.; Gan et al., 1998, J. Med. Coll. PLA, 13(3), 157-159.; Li et al., 1999, Jiefangjun Yixue Zazhi, 24(2), 99-101; Putlitz et al., 1999, J. Virol., 73(7), 5381-5387.; Kim et al., 1999, Biochem. Biophys. Res. Commun., 257(3), 759-765.; Xu et al., 1998, Bingdu Xuebao, 14(4), 365-369.; Welch et al., 1997, Gene Ther., 4(7), 736-743.; Goldenberg et al., 1997, International PCT publication No. WO 97/08309, Wands et al., 1997, J. of Gastroenterology and Hepatology, 12(suppl.), S354-S369.; Ruiz et al., 1997, BioTechniques, 22(2), 338-345.; Gan et al., 1996, J. Med. Coll. PLA, 11(3), 171-175.; Beck and Nassal, 1995, Nucleic Acids Res., 23(24), 4954-62.; Goldenberg, 1995, International PCT publication No. WO 95/22600.; Xu et al., 1993, Bingdu Xuebao, 9(4), 331-6.; Wang et al., 1993, Bingdu Xuebao, 9(3), 278-80, all describe ribozymes that are targeted to cleave a specific HBV target site.

Hunt et al., U.S. Pat. No. 5,859,226, describes specific non-naturally occurring oligonucleotide decoys intended to inhibit the expression of MHC-II genes through binding of the RF-X transcription factor, that can inhibit the expression of certain HBV and CMV viral proteins.

Kao et al., International PCT Publication No. WO 00/04141, describes linear single stranded nucleic acid molecules capable of specifically binding to viral polymerases and inhibiting the activity of the viral polymerase.

Lu, International PCT Publication No. WO 99/20641, describes specific triplex-forming oligonucleotides used in treating HBV infection.

### SUMMARY OF THE INVENTION

This invention relates to nucleic acid molecules directed to disrupt the function of HBV reverse transcriptase. The invention also relates to nucleic acid molecules directed to disrupt the function of the Enhancer I core region of the HBV genomic DNA. In particular, the present invention describes the selection and function of nucleic acid molecules, such as decoys and aptamers, capable of specifically binding to the HBV reverse transcriptase (pol) primer and modulating reverse transcription of the HBV pregenomic RNA. In another embodiment, the present invention relates to nucleic acid molecules, such as decoys, antisense and aptamers, capable of specifically binding to the HBV reverse transcriptase (pol) and modulating reverse transcription of the HBV pregenomic RNA. In yet another embodiment, the present invention relates to nucleic acid molecules capable of specifically binding to the HBV Enhancer I core region and modulating transcription of the HBV genomic DNA. The invention further relates to allosteric enzymatic nucleic acid molecules or “allozymes” that are used to modulate HBV gene expression. Such allozymes are active in the presence of HBV-derived nucleic acids, peptides, and/or proteins such as HBV reverse transcriptase and/or a HBV reverse transcriptase primer sequence, thereby allowing the allozyme to selectively cleave a sequence of HBV DNA or RNA. Allozymes of the invention are also designed to be active in the presence of HBV Enhancer I sequences and/or mutant HBV Enhancer I sequences, thereby allowing the allozyme to selectively cleave a sequence of HBV DNA or RNA. These nucleic acid molecules can be used to treat diseases and disorders associated with HBV infection.

In one embodiment, the invention features a nucleic acid decoy molecule that specifically binds the hepatitis B virus (HBV) reverse transcriptase primer sequence. In another embodiment, the invention features a nucleic acid decoy molecule that specifically binds the hepatitis B virus (HBV) reverse transcriptase. In yet another embodiment, the invention features a nucleic acid decoy molecule that specifically binds to the HBV Enhancer I core sequence.

In one embodiment, the invention features a nucleic acid aptamer that specifically binds the hepatitis B virus (HBV) reverse transcriptase primer. In another embodiment, the invention features a nucleic acid aptamer that specifically binds the hepatitis B virus (HBV) reverse transcriptase. In yet another embodiment, the invention features a nucleic acid aptamer molecule that specifically binds to the HBV Enhancer I core sequence.

In one embodiment, the invention features an allozyme that specifically binds the hepatitis B virus (HBV) reverse transcriptase primer. In another embodiment, the invention features an allozyme that specifically binds the hepatitis B virus (HBV) reverse transcriptase. In yet another embodiment, the invention features an allozyme that specifically binds to the HBV Enhancer I core sequence.

In yet another embodiment, the invention features a nucleic acid molecule, for example a triplex forming nucleic acid molecule or antisense nucleic acid molecule, that binds the hepatitis B virus (HBV) reverse transcriptase primer. In another embodiment, the invention features a triplex forming nucleic acid molecule or antisense nucleic acid molecule that specifically binds the hepatitis B virus (HBV) reverse transcriptase. In yet another embodiment, the invention features a triplex forming nucleic acid molecule or antisense nucleic acid molecule that specifically binds to the HBV Enhancer I core sequence.

In another embodiment, a nucleic acid molecule of the invention binds to Hepatocyte Nuclear Factor 3 (HNF3) and/or Hepatocyte Nuclear Factor 4 (HNF4) binding sequence within the HBV Enhancer I region of HBV genomic DNA, for example the plus strand and/or minus strand DNA of the Enhancer I region, and blocks the binding of HNF3 and/or HNF4 to the Enhancer 1 region.

In another embodiment, the nucleic acid molecule of the invention comprises a sequence having (UUCA)n domain, where n is an integer from 1-10. In another embodiment, the nucleic acid molecules of the invention comprise the sequence of SEQ. ID NOs: 1-128.

In another embodiment, the invention features a pharmaceutical composition comprising a nucleic acid molecule of the invention in a pharmaceutically acceptable carrier. In another embodiment, the invention features a mammalian cell, for example a human cell, including a nucleic acid molecule contemplated by the invention.

In one embodiment, the invention features a method for treatment of HBV infection, cirrhosis, liver failure, or hepatocellular carcinoma, comprising administering to a subject a nucleic acid molecule of the invention under conditions suitable for the treatment.

In another embodiment, the invention features a method of treatment of a subject having a condition associated with HBV infection, comprising contacting cells of said subject with a nucleic acid molecule of the invention under conditions suitable for such treatment. In another embodiment, the invention features a method of treatment of a subject having a condition associated with HBV infection, comprising contacting cells of said subject with a nucleic acid molecule of the invention, and further comprising the use of one or more drug therapies, for example type I interferon or 3TC® (lamivudine), under conditions suitable for said treatment. In another embodiment, the other therapy is administered simultaneously with or separately from the nucleic acid molecule.

In another embodiment, the invention features a method for modulating HBV replication in a mammalian cell comprising administering to the cell a nucleic acid molecule of the invention under conditions suitable for the modulation.

In yet another embodiment, the invention features a method of modulating HBV reverse transcriptase activity comprising contacting HBV reverse transcriptase with a nucleic acid molecule of the invention, for example a decoy or aptamer, under conditions suitable for the modulation of the HBV reverse transcriptase activity.

In another embodiment, the invention features a method of modulating HBV transcription, comprising contacting an HBV Enhancer I sequence with a nucleic molecule of the invention with under conditions suitable for the modulation of HBV transcription.

In one embodiment, a nucleic acid molecule of the invention, for example a decoy or aptamer, is chemically synthesized. In another embodiment, the nucleic acid molecule of the invention comprises at least one nucleic acid sugar modification. In yet another embodiment, the nucleic acid molecule of the invention comprises at least one nucleic acid base modification. In another embodiment, the nucleic acid molecule of the invention comprises at least one nucleic acid backbone modification.

In another embodiment, the nucleic acid molecule of the invention comprises at least one 2′-O-alkyl, 2′-alkyl, 2′-alkoxylalkyl, 2′-alkylthioalkyl, 2′-amino, 2′-O-amino, or 2′-halo modification and/or any combination thereof with or without 2′-deoxy and/or 2′-ribo nucleotides. In yet another embodiment, the nucleic acid molecule of the invention comprises all 2′-O-alkyl nucleotides, for example, all 2′-O-allyl nucleotides.

In one embodiment, the nucleic acid molecule of the invention comprises a 5′-cap, 3′-cap, or 5′-3′ cap structure, for example an abasic or inverted abasic moiety.

In another embodiment, the nucleic acid molecule of the invention is a linear nucleic acid molecule. In another embodiment, the nucleic acid molecule of the invention is a linear nucleic acid molecule that can optionally form a hairpin, loop, stem-loop, or other secondary structure. In yet another embodiment, the nucleic acid molecule of the invention is a circular nucleic acid molecule.

In one embodiment, the nucleic acid molecule of the invention is a single stranded oligonucleotide. In another embodiment, the nucleic acid molecule of the invention is a double-stranded oligonucleotide.

In one embodiment, the nucleic acid molecule of the invention comprises an oligonucleotide having about 3 to about 100 nucleotides. In another embodiment, the nucleic acid molecule of the invention comprises an oligonucleotide having about 3 to about 24 nucleotides. In another embodiment, the nucleic acid molecule of the invention comprises an oligonucleotide having about 4 to about 16 nucleotides.

In another embodiment, the nucleic acid molecule of the invention binds irreversibly to the HBV reverse transcriptase target, for example, by covalent attachment of the nucleic acid molecule to the reverse transcriptase primer sequence. The covalent attachment can be accomplished by introducing chemical modifications into the sequence of the nucleic acid molecule (for example, decoy or aptamer) that are capable of forming covalent bonds with the reverse transcriptase primer sequence.

In another embodiment, the nucleic acid molecule of the invention binds irreversibly to the HBV Enhancer I sequence target, for example, by covalent attachment of the nucleic acid molecule to the HBV Enhancer I sequence. The covalent attachment can be accomplished by introducing chemical modifications into the sequence of the nucleic acid moleculethat are capable of forming covalent bonds with the Enhancer I sequence.

In another embodiment, the type I interferon contemplated by the invention is interferon alpha, interferon beta, consensus interferon, polyethylene glycol interferon, polyethylene glycol interferon alpha 2a, polyethylene glycol interferon alpha 2b, or polyethylene glycol consensus interferon.

In one embodiment, the invention features a pharmaceutical composition comprising type I interferon and a nucleic acid molecule of the invention in a pharmaceutically acceptable carrier.

In another embodiment, the invention features a method of administering a nucleic acid molecule of the invention to a cell, for example a mammalian cell or human cell, comprising contacting the cell with the nucleic acid molecule under conditions suitable for the administration. The nucleic acid molecule can be administered independently or in conjunction with other therapeutic compounds, such as type I interferon or 3TC® (lamivudine).

In yet another embodiment, the invention features a method of administering a nucleic acid molecule of the invention to a cell, for example a mammalian cell or human cell, comprising contacting the cell with the nucleic acid molecule of the invention under conditions suitable for the administration. independently or in conjunction with other therapeutic compounds, such as enzymatic nucleic acid molecules, antisense molecules, triplex forming oligonucleotides, 2,5-A chimeras, and/or RNAi.

In another embodiment, a nucleic acid molecule of the invention is administered to a cell or subject in the presence of a delivery reagent, for example a lipid, cationic lipid, phospholipid, or liposome.

In one embodiment, the invention features novel nucleic acid-based molecules and techniques, such as nucleic acid decoy molecules and/or aptamers, used alone or in combination with enzymatic nucleic acid molecules, antisense molecules, and/or RNAi, and methods for use to down regulate or modulate the expression of HBV RNA and/or replication of HBV.

In another embodiment, the invention features the use of one or more of the nucleic acid-based molecules and techniques to modulate the expression of genes encoding HBV viral proteins. Specifically, the invention features the use of nucleic acid-based molecules and techniques to specifically modulate the expression of the HBV viral genome.

In another embodiment, the invention features the use of one or more of the nucleic acid-based molecules and techniques to modulate the activity, expression, or level of cellular proteins required for HBV replication. For example, the invention features the use of nucleic acid-based molecules and techniques to specifically modulate the activity of cellular proteins required for HBV replication.

In another embodiment, the invention features a nucleic acid sensor molecule having an enzymatic nucleic acid domain and a sensor domain that interacts with an HBV peptide, protein, or polynucleotide sequence, for example, HBV reverse transcriptase, HBV reverse transcriptase primer, or the Enhancer I element of the HBV pregenomic RNA, wherein such interaction results in modulation of the activity of the enzymatic nucleic acid domain of the nucleic acid sensor molecule. The invention features HBV-specific nucleic acid sensor molecules or allozymes, and methods for their use to down regulate or modulate the expression of HBV RNA capable of progression and/or maintenance of hepatitis, hepatocellular carcinoma, cirrhosis, and/or liver failure. In one embodiment, the enzymatic nucleic acid domain of a nucleic acid sensor molecule of the invention is a Hammerhead, Inozyme, G-cleaver, DNAzyme, Zinzyme, Amberzyme, or Hairpin enzymatic nucleic acid molecule.

In one embodiment, one or more nucleic acid molecules of the invention are used to treat HBV-infected cells or an HBV-infected subject wherein the HBV is resistant or the subject does not respond to treatment with 3TC® (Lamivudine). The nucleic molecule(s) can be used either alone or in combination with other therapies under conditions suitable for the treatment.

In another embodiment, one or more nucleic acid molecules of the invention are used to treat HBV-infected cells or an HBV-infected subject, wherein the HBV is resistant or the subject does not respond to treatment with Interferon, for example Infergen®. The nucleic molecule(s) can be used either alone or in combination with other therapies under conditions suitable for the treatment.

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit” it is meant that the activity of HBV, HBV reverse transcriptase, or level of RNAs or equivalent RNAs encoding one or more protein subunits of HBV is reduced below that observed in the absence of the nucleic acid of the invention. In one embodiment, inhibition with nucleic acid decoy molecule preferably is below that level observed in the presence of an inactive or attenuated molecule that is unable to bind to the same site on the viral polymerase. In another embodiment, inhibition of HBV reverse transcriptase with the nucleic acid molecule of the instant invention is greater in the presence of the nucleic acid molecule than in its absence.

The methods of this invention can be used to treat human hepatitis B virus infections, which include, for example, productive virus infection, latent or persistent virus infection, and HBV-induced hepatocyte transformation. The utility can be extended to other species of HBV that infect non-human animals where such infections are of veterinary importance.

Preferred binding sites of the nucleic acid molecules of the invention include, but are not limited to, the primer binding site on HBV reverse transcriptase, the primer binding sequences of the HBV RNA, and/or the HBV Enhancer I region of HBV DNA.

By “nucleic acid decoy molecule” or “decoy” as used herein is meant a nucleic acid molecule that mimics the natural binding domain for a ligand. The decoy therefore competes with the natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). Similarly, the nucleic acid molecules of the instant invention can bind to HBV reverse transcriptase, HBV reverse transcriptase primer, or Enhancer I element of the HBV pregenomic RNA to block transcription of the HBV genome.

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

By “enzymatic nucleic acid molecule” is meant a nucleic acid molecule that has complementarity in a substrate binding region to a specified gene target, and also has an enzymatic activity which is active to specifically cleave a target RNA molecule. That is, the enzymatic nucleic acid molecule is able to intermolecularly cleave a RNA molecule and thereby inactivate a target RNA molecule. These complementary regions allow sufficient hybridization of the enzymatic nucleic acid molecule to a target RNA molecule and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA 260:20 3030-4).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof.

By “Inozyme” or “NCH” motif or configuration is meant, an enzymatic nucleic acid molecule comprising a motif as is generally described as NCH Rz in Ludwig et al., International PCT Publication No. WO 98/58058 and U.S. patent application Ser. No. 08/878,640. Inozymes possess endonuclease activity to cleave RNA substrates having a cleavage triplet NCH/, where N is a nucleotide, C is cytidine and H is adenosine, uridine or cytidine, and/represents the cleavage site. Inozymes can also possess endonuclease activity to cleave RNA substrates having a cleavage triplet NCN/, where N is a nucleotide, C is cytidine, and/represents the cleavage site

By “G-cleaver” motif or configuration is meant, an enzymatic nucleic acid molecule comprising a motif as is generally described in Eckstein et al., U.S. Pat. No. 6,127,173 and in Kore et al., 1998, Nucleic Acids Research 26, 4116-4120. G-cleavers possess endonuclease activity to cleave RNA substrates having a cleavage triplet NYN/, where N is a nucleotide, Y is uridine or cytidine and/represents the cleavage site. G-cleavers can be chemically modified.

By “zinzyme” motif or configuration is meant, an enzymatic nucleic acid molecule comprising a motif as is generally described in Beigelman et al., International PCT publication No. WO 99/55857 and U.S. patent application Ser. No. 09/918,728. Zinzymes possess endonuclease activity to cleave RNA substrates having a cleavage triplet including but not limited to, YG/Y, where Y is uridine or cytidine, and G is guanosine and/represents the cleavage site. Zinzymes can be chemically modified to increase nuclease stability through various substitutions, including substituting 2′-O-methyl guanosine nucleotides for guanosine nucleotides. In addition, differing nucleotide and/or non-nucleotide linkers can be used to substitute the 5′-gaaa-2′ loop of the motif. Zinzymes represent a non-limiting example of an enzymatic nucleic acid molecule that does not require a ribonucleotide (2′-OH) group within its own nucleic acid sequence for activity.

By “amberzyme” motif or configuration is meant, an enzymatic nucleic acid molecule comprising a motif as is generally described in Beigelman et al., International PCT publication No. WO 99/55857 and U.S. patent application Ser. No. 09/476,387. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability. In addition, differing nucleoside and/or non-nucleoside linkers can be used to substitute the 5′-gaaa-3′ loops of the motif. Amberzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments, the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. Non-limiting examples of DNAzymes are generally reviewed in Usman et al., U.S. Pat. No. 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection as generally described in Joyce et al., U.S. Pat. No. 5,807,718 and Santoro et al., supra. Additional DNAzyme motifs can be selected for using techniques similar to those described in these references, and hence, are within the scope of the present invention.

By “nucleic acid sensor molecule” or “allozyme” as used herein is meant a nucleic acid molecule comprising an enzymatic domain and a sensor domain, where the enzymatic nucleic acid domain's ability to catalyze a chemical reaction is dependent on the interaction with a target signaling molecule, such as a nucleic acid, polynucleotide, oligonucleotide, peptide, polypeptide, or protein, for example HBV RT, HBV RT primer, or HBV Enhancer I sequence. The introduction of chemical modifications, additional functional groups, and/or linkers, to the nucleic acid sensor molecule can provide enhanced catalytic activity of the nucleic acid sensor molecule, increased binding affinity of the sensor domain to a target nucleic acid, and/or improved nuclease/chemical stability of the nucleic acid sensor molecule, and are hence within the scope of the present invention (see for example Usman et al., U.S. patent application Ser. No. 09/877,526, George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., U.S. patent application Ser. No. 09/205,520).

By “sensor component” or “sensor domain” of the nucleic acid sensor molecule as used herein is meant, a nucleic acid sequence (e.g., RNA or DNA or analogs thereof) which interacts with a target signaling molecule, for example a nucleic acid sequence in one or more regions of a target nucleic acid molecule or more than one target nucleic acid molecule, and which interaction causes the enzymatic nucleic acid component of the nucleic acid sensor molecule to either catalyze a reaction or stop catalyzing a reaction. In the presence of a target signaling molecule of the invention, such as HBV RT, HBV RT primer, or HBV Enhancer I sequence, the ability of the sensor component, for example, to modulate the catalytic activity of the nucleic acid sensor molecule, is modulated or diminished. The sensor component can comprise recognition properties relating to chemical or physical signals capable of modulating the nucleic acid sensor molecule via chemical or physical changes to the structure of the nucleic acid sensor molecule. The sensor component can be derived from a naturally occurring nucleic acid binding sequence, for example, RNAs that bind to other nucleic acid sequences in vivo. Alternately, the sensor component can be derived from a nucleic acid molecule (aptamer), which is evolved to bind to a nucleic acid sequence within a target nucleic acid molecule. The sensor component can be covalently linked to the nucleic acid sensor molecule, or can be non-covalently associated. A person skilled in the art will recognize that all that is required is that the sensor component is able to selectively modulate the activity of the nucleic acid sensor molecule to catalyze a reaction.

By “target molecule” or “target signaling molecule” is meant a molecule capable of interacting with a nucleic acid sensor molecule, specifically a sensor domain of a nucleic acid sensor molecule, in a manner that causes the nucleic acid sensor molecule to be active or inactive. The interaction of the signaling agent with a nucleic acid sensor molecule can result in modification of the enzymatic nucleic acid component of the nucleic acid sensor molecule via chemical, physical, topological, or conformational changes to the structure of the molecule, such that the activity of the enzymatic nucleic acid component of the nucleic acid sensor molecule is modulated, for example is activated or deactivated. Signaling agents can comprise target signaling molecules such as macromolecules, ligands, small molecules, metals and ions, nucleic acid molecules including but not limited to RNA and DNA or analogs thereof, proteins, peptides, antibodies, polysaccharides, lipids, sugars, microbial or cellular metabolites, pharmaceuticals, and organic and inorganic molecules in a purified or unpurified form, for example HBV RT or HBV RT primer.

By “sufficient length” is meant a nucleic acid molecule long enough to provide the intended function under the expected condition. For example, a nucleic acid molecule of the invention needs to be of “sufficient length” to provide stable binding to a target site under the expected binding conditions and environment. In another non-limiting example, for the binding arms of an enzymatic nucleic acid, “sufficient length” means that the binding arm sequence is long enough to provide stable binding to a target site under the expected reaction conditions and environment. The binding arms are not so long as to prevent useful turnover of the nucleic acid molecule. By “stably interact” is meant interaction of the oligonucleotides with target nucleic acid (e.g., by forming hydrogen bonds with complementary nucleotides in the target under physiological conditions) that is sufficient for the intended purpose (e.g., cleavage of target RNA by an enzyme).

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al, U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two or more non-contiguous substrate sequences or two or more non-contiguous sequence portions of an antisense molecule can be complementary to a target sequence, or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. Antisense molecules of the instant invention can include 2-5A antisense chimera molecules. In addition, antisense DNA can be used to target RNA by means of DNA-RNA interactions, thereby activating RNase H, which digests the target RNA in the duplex. The antisense oligonucleotides can comprise one or more RNAse H activating region that is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (for example, at least four of the nucleotides are phosphorothiote substitutions; more specifically, 4-11 of the nucleotides are phosphorothiote substitutions), phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-SA antisense chimera” it is meant, an antisense oligonucleotide containing a 5′-phosphorylated 2′-5′-linked adenylate residue. These chimeras bind to target RNA in a sequence-specific manner and activate a cellular 2-SA-dependent ribonuclease, which, in turn, cleaves the target RNA (Torrence et al., 1993 Proc. Natl. Acad. Sci. USA 90, 1300).

By “triplex nucleic acid” or “triplex oligonucleotide” it is meant a polynucleotide or oligonucleotide that can bind to a double-stranded DNA in a sequence-specific manner to form a triple-strand helix. Formation of such triple helix structure has been shown to modulate transcription of the targeted gene (Duval-Valentin et al., 1992, Proc. Natl. Acad. Sci. USA, 89, 504). Triplex nucleic acid molecules of the invention also include steric blocker nucleic acid molecules that bind to the Enhancer I region of HBV DNA (plus strand and/or minus strand) and prevent translation of HBV genomic DNA.

The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

By “gene” it is meant a nucleic acid that encodes an RNA, for example, nucleic acid sequences including, but not limited to, structural genes encoding a polypeptide.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., ribozyme cleavage, antisense or triple helix modulation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The nucleic acid decoy molecules and/or aptamers that bind to a reverse transcriptase and/or reverse transcriptase primer and therefore inactivate the reverse transcriptase, represent a novel therapeutic approach to treat a variety of pathologic indications, including, viral infection such as HBV infection, hepatitis, hepatocellular carcinoma, tumorigenesis, cirrhosis, liver failure and others.

The nucleic acid molecules that bind to a HBV Enhancer I sequence and therefore inactivate HBV transcription, represent a novel therapeutic approach to treat a variety of pathologic indications, including viral infection, such as HBV infection, hepatitis, hepatocellular carcinoma, tumorigenesis, cirrhosis, liver failure and others.

In one embodiment of the present invention, a decoy nucleic acid molecule of the invention is 4 to 50 nucleotides in length, in specific embodiments about 4, 5, 6, 7, 8, 9, 10, 11, 12, 13, 14, 15, or 16 nucleotides in length. In another embodiment, a non-decoy nucleic acid molecule, e.g., an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

Exemplary nucleic acid decoy molecules of the invention are shown in Table I. Exemplary synthetic nucleic acid molecules of the invention are shown in Table II. For example, decoy molecules of the invention are about 4 to about 50 nucleotides in length. Exemplary decoys of the invention are 4, 8, 12, or 16 nucleotides in length. In an additional example, enzymatic nucleic acid molecules of the invention are about 15 to about 50 nucleotides in length, for example, 25 to 40 nucleotides in length, e.g., 34, 36, or 38 nucleotides in length (for example see Jarvis et al., 1996, J. Biol. Chem., 271, 29107-29112). Exemplary DNAzymes of the invention are about 15 to about 40 nucleotides in length, for example, 25 to 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are about 15 to about 75 nucleotides in length, for example, 20 to 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are about 10 to about 40 nucleotides in length, for example, 12 to 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is that the nucleic acid molecule is of length and conformation sufficient and suitable for the nucleic acid molecule to catalyze a reaction contemplated herein. The length of the nucleic acid molecules of the instant invention are not limiting within the general limits stated.

In one embodiment, the invention provides a method for producing a class of nucleic acid-based gene modulating agents, which exhibit a high degree of specificity for a viral reverse transcriptase, such as HBV reverse transcriptase, or reverse transcriptase primer, such as a HBV reverse transcriptase primer. For example, the nucleic acid molecule can be targeted to a highly conserved nucleic acid binding region of the viral reverse transcriptase such that specific treatment of a disease or condition can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

In another embodiment, the invention provides a method for producing a class of nucleic acid-based gene modulating agents which exhibit a high degree of specificity for a viral enhancer regions, such as the HBV Enhancer I core sequence. For example, the nucleic acid molecule can be targeted to a highly conserved transcription factor-binding region of the viral Enhancer I sequence such that specific treatment of a disease or condition can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “HBV proteins” is meant, a protein or a mutant protein derivative thereof, comprising sequence expressed and/or encoded by the HBV genome.

By “highly conserved nucleic acid binding region” is meant an amino acid sequence of one or more regions in a target protein that does not vary significantly from one generation to the other or from one biological system to the other.

The enzymatic nucleic acid-based modulators of HBV expression are useful for the prevention of the diseases and conditions including HBV infection, hepatitis, cancer, cirrhosis, liver failure, and any other diseases or conditions that are related to the levels of HBV in a cell or tissue.

By “related to the levels of HBV” is meant that the reduction of HBV expression or HBV RNA or protein levels will relieve, to some extent, the symptoms of the disease or condition.

The nucleic acid-based modulators of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Table I and Table II. Examples of such nucleic acid molecules consist essentially of sequences defined in the tables.

In another aspect, the invention provides mammalian cells comprising one or more nucleic acid molecules and/or expression vectors of this invention. The one or more nucleic acid molecules can independently be targeted to the same or different sites.

In another aspect of the invention, nucleic acid decoys, aptamers, enzymatic nucleic acids or antisense molecules that interact with target protein and/or RNA molecules and modulate HBV (for example, HBV reverse transcriptase, or transcription of HBV genomic DNA) activity are expressed from transcription units inserted into DNA or RNA vectors. The recombinant vectors are preferably DNA plasmids or viral vectors. Decoys, aptamers, enzymatic nucleic acid or antisense expressing viral vectors can be constructed based on, but not limited to, adeno-associated virus, retrovirus, adenovirus, or alphavirus. Preferably, the recombinant vectors capable of expressing the decoys, aptamers, enzymatic nucleic acids or antisense are delivered as described above, and persist in target cells. Alternatively, viral vectors can be used that provide for transient expression of decoys, aptamers, enzymatic nucleic acids or antisense. Such vectors might be repeatedly administered as necessary. Once expressed, the decoys, aptamers, enzymatic nucleic acids or antisense bind to the target protein and/or RNA and modulate its function or expression. Delivery of decoy, aptamer, enzymatic nucleic acid or antisense expressing vectors can be systemic, such as by intravenous or intramuscular administration, by administration to target cells ex-planted from the subject followed by reintroduction into the subject, or by any other means that would allow for introduction into the desired target cell. DNA based nucleic acid molecules of the invention can be expressed via the use of a single stranded DNA intracellular expression vector.

By RNA is meant a molecule comprising at least one ribonucleotide residue. By “ribonucleotide” is meant a nucleotide with a hydroxyl group at the 2′ position of a β-D-ribo-furanose moiety.

By “vectors” is meant any nucleic acid- and/or viral-based technique used to express and/or deliver a desired nucleic acid.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

The nucleic acid molecules of the instant invention, individually, or in combination or in conjunction with other drugs, can be used to treat diseases or conditions discussed herein. For example, to treat a disease or condition associated with the levels of HBV, the nucleic acid molecules can be administered to a subject or can be administered to other appropriate cells evident to those skilled in the art, individually or in combination with one or more drugs under conditions suitable for the treatment.

In a further embodiment, the described molecules, such as decoys, aptamers, antisense, or enzymatic nucleic acids, can be used in combination with other known treatments to treat conditions or diseases discussed above. For example, the described molecules can be used in combination with one or more known therapeutic agents to treat a condition related to HBV levels, such as HBV infection, hepatitis, hepatocellular carcinoma, cancer, cirrhosis, and liver failure. Such therapeutic agents include, but are not limited to, nucleoside analogs selected from the group comprising Lamivudine (3TC®), L-FMAU, and/or adefovir dipivoxil (for a review of applicable nucleoside analogs, see Colacino and Staschke, 1998, Progress in Drug Research, 50, 259-322). Immunomodulators selected from the group comprising Type 1 Interferon, therapeutic vaccines, steriods, and 2′-5′ oligoadenylates (for a review of 2′-5′ Oligoadenylates, see Charubala and Pfleiderer, 1994, Progress in Molecular and Subcellular Biology, 14, 113-138).

In another embodiment, the invention features nucleic acid-based modulators (e.g., nucleic acid decoy molecules, aptamers, enzymatic nucleic acid molecules (ribozymes), antisense nucleic acids, triplex DNA, antisense nucleic acids containing RNA cleaving chemical groups) and methods for their use to down regulate or modulate reverse transcriptase activity and/or the expression of RNA (e.g., HBV) capable of progression and/or maintenance of HBV infection, hepatocellular carcinoma, liver disease and failure.

In another embodiment, the invention features nucleic acid-based molecules and techniques (e.g., nucleic acid decoy molecules, aptamers, enzymatic nuleic acid molecules (ribozymes), antisense nucleic acid molecules, triplex DNA, antisense nucleic acids containing RNA cleaving chemical groups) and methods for their use to down regulate or modulate reverse transcriptase activity and/or the expression of HBV RNA.

In another embodiment, the invention features nucleic acid-based modulators (e.g., nucleic acid decoy molecules, aptamers, enzymatic nucleic acid molecules (ribozymes), antisense nucleic acids, triplex DNA, siRNA, dsRNA, antisense nucleic acids containing RNA cleaving chemical groups) and methods for their use to down regulate or modulate Enhancer I mediated transcription activity and/or the expression of DNA (e.g., HBV) capable of progression and/or maintenance of HBV infection, hepatocellular carcinoma, liver disease and failure.

In another embodiment, the invention features nucleic acid-based molecules and techniques (e.g., nucleic acid decoy molecules, aptamers, enzymatic nuleic acid molecules, antisense nucleic acid molecules, triplex DNA, antisense nucleic acids containing DNA cleaving chemical groups) and methods for their use to down regulate or modulate Enhancer I mediated transcription activity and/or the expression of HBV DNA.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 is a schematic diagram which outlines the steps involved in HBV reverse transcription. The HBV polymerase/reverse transcriptase binds to the 5′-stem-loop of the HBV pregenomic RNA and synthesizes a primer from the UUCA template. The reverse transcriptase and tetramer primer are translocated to the 3′-DR1 site. The RT primer binds to the UUCA sequence in the DR1 element and minus strand synthesis begins.

FIG. 2 is a schematic diagram showing a non-limiting example of inhibition of HBV reverse transcription. In this example, a decoy molecule binds to the HBV RT primer, thereby preventing translocation of the RT to the 3′-DR1 site and preventing minus strand synthesis.

FIG. 3 is a graphical representation of HbsAg levels in the presence of different 2′-O-allyl modified nucleic acid molecules as determined by ELISA. Inhibition of HBV is correlated to HBsAg antigen levels.

FIG. 4 is a graphical representation of HbsAg levels in the presence of different 2′-O-methyl modified nucleic acid molecules as determined by ELISA. Inhibition of HBV is correlated to HBsAg antigen levels.

FIG. 5 is a graphical representation of HbsAg levels in the presence of various concentrations (100-800 nM) of inverse control nucleic acid molecules and 2′-O-methyl modified nucleic acid molecules targeted to the HBV reverse transcriptase primer.

FIG. 6 is a graphical representation of HbsAg levels in the presence of nucleic acid molecules (200 nM) targeted to the HBV Enhancer I core region.

FIG. 7 is a graphical representation of HbsAg levels in the presence of nucleic acid molecules (400 nM) targeted to the HBV Enhancer I core region.

FIG. 8 is a graphical representation of HbsAg levels in the presence of various concentrations (50-200 nM) of inverse control nucleic acid molecules and nucleic acid molecules targeted to the HBV Enhancer I core region.

### MECHANISM OF ACTION OF NUCLEIC ACID MOLECULES OF THE INVENTION

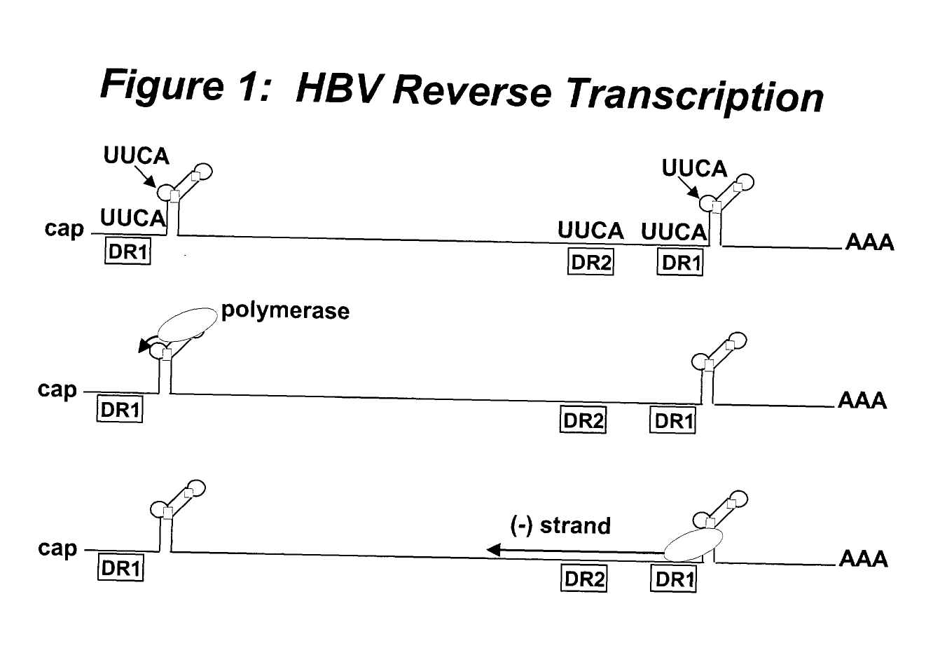

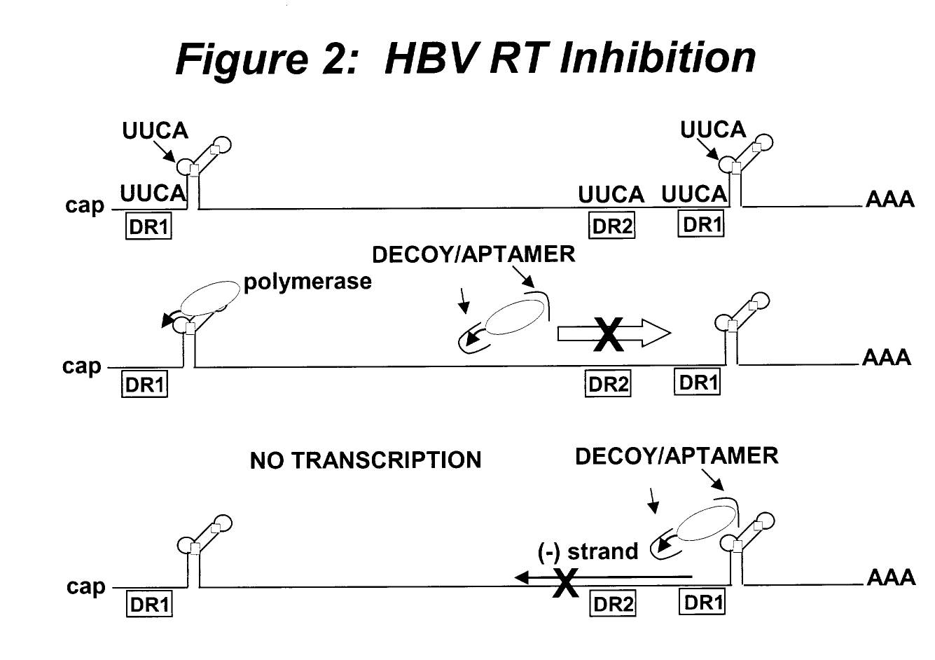

Decoy: Nucleic acid decoy molecules are mimetics of naturally occurring nucleic acid molecules or portions of naturally occurring nucleic acid molecules that can be used to modulate the function of a specific protein or a nucleic acid whose activity is dependant on interaction with the naturally occurring nucleic acid molecule. Decoys modulate the function of a target protein or nucleic acid by competing with authentic nucleic acid binding to the ligand of interest. Often, the nucleic acid decoy is a truncated version of a nucleic acid sequence that is recognized, for example by a particular protein, such as a transcription factor or polymerase. Decoys can be chemically modified to increase binding affinity to the target ligand as well as to increase the enzymatic and chemical stability of the decoy. In addition, bridging and non-bridging linkers can be introduced into the decoy sequence to provide additional binding affinity to the target ligand. Decoy molecules of the invention that bind to a reverse transcriptase or reverse transcriptase primer, for example, HBV reverse transcriptase or HBV reverse transcriptase primer, or an enhancer region of the HBV pregenomic RNA, for example, the Enhancer I element, modulate the transcription of RNA to DNA and therefore modulate expression of the pregenomic RNA of the virus (see FIGS. 1-2).

Aptamer: Nucleic acid aptamers can be selected to specifically bind to a particular ligand of interest (see for example Gold et al., U.S. Pat. No. 5,567,588 and U.S. Pat. No. 5,475,096, Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628). For example, the use of in vitro selection can be applied to evolve nucleic acid aptamers with binding specificity for HBV RT and/or HBV RT primer. Nucleic acid aptamers can include chemical modifications and linkers as described herein. Aptamer molecules of the invention that bind to a reverse transcriptase or reverse transcriptase primer, such as HBV reverse transcriptase or HBV reverse transcriptase primer, modulate the transcription of RNA to DNA and therefore modulate expression of the pregenomic RNA of the virus.

Antisense: Antisense molecules can be modified or unmodified RNA, DNA, or mixed polymer oligonucleotides and primarily function by specifically binding to matching sequences resulting in modulation of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently, it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. S No. 60/082,404 which was filed on Apr. 20, 1998; Hartmann et al., U.S. S No. 60/101,174 which was filed on Sep. 21, 1998) all of these are incorporated by reference herein in their entirety.

Antisense DNA can be used to target RNA by means of DNA-RNA interactions, thereby activating RNase H, which digests the target RNA in the duplex. Antisense DNA can be chemically synthesized or can be expressed via the use of a single stranded DNA intracellular expression vector or the equivalent thereof.

Triplex Forming Oligonucleotides (TFO): Single stranded oligonucleotide can be designed to bind to genomic DNA in a sequence specific manner. TFOs can be comprised of pyrimidine-rich oligonucleotides which bind DNA helices through Hoogsteen Base-pairing (Wu-Pong, supra). In addition, TFOs can be chemically modified to increase binding affinity to target DNA sequences. The resulting triple helix composed of the DNA sense, DNA antisense, and TFO disrupts RNA synthesis by RNA polymerase. The TFO mechanism can result in gene expression or cell death since binding may be irreversible (Mukhopadhyay & Roth, supra)

2′-5′ Oligoadenylates: The 2-5A system is an interferon-mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L, which has the ability to cleave single stranded RNA. The ability to form 2-5A structures with double stranded RNA makes this system particularly useful for modulation of viral replication.

(2′-5′) oligoadenylate structures can be covalently linked to antisense molecules to form chimeric oligonucleotides capable of RNA cleavage (Torrence, supra). These molecules putatively bind and activate a 2-5A-dependent RNase, the oligonucleotide/enzyme complex then binds to a target RNA molecule which can then be cleaved by the RNase enzyme. The covalent attachment of 2′-5′ oligoadenylate structures is not limited to antisense applications, and can be further elaborated to include attachment to nucleic acid molecules of the instant invention.

Enzymatic Nucleic Acid: Several varieties of naturally occurring enzymatic RNAs are presently known (Doherty and Doudna, 2001, Annu. Rev. Biophys. Biomol. Struct., 30, 457-475; Symons, 1994, Curr. Opin. Struct. Biol., 4, 322-30). In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J, 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495). Each can catalyze a series of reactions including the hydrolysis of phosphodiester bonds in trans (and thus can cleave other RNA molecules) under physiological conditions.

Nucleic acid molecules of this invention can block HBV protein expression and can be used to treat disease or diagnose disease associated with the levels of HBV.

The enzymatic nature of an enzymatic nucleic acid has significant advantages, such as the concentration of nucleic acid necessary to affect a therapeutic treatment is low. This advantage reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific modulator, with the specificity of modulation depending not only on the base-pairing mechanism of binding to the target RNA, but also on the mechanism of target RNA cleavage. Single mismatches, or base-substitutions, near the site of cleavage can be chosen to completely eliminate catalytic activity of an enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to any RNA transcript, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Chartrand et al., 1995, Nucleic Acids Research 23, 4092; Santoro et al., 1997, PNAS 94, 4262).

Because of their sequence specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecule can be designed to cleave specific RNA targets within the background of cellular RNA. Such a cleavage event renders the RNA non-functional and abrogates protein expression from that RNA. In this manner, synthesis of a protein associated with a disease state can be selectively modulated (Warashina et al., 1999, Chemistry and Biology, 6, 237-250.

The present invention also features nucleic acid sensor molecules or allozymes having sensor domains comprising nucleic acid decoys and/or aptamers of the invention. Interaction of the nucleic acid sensor molecule's sensor domain with a molecular target, such as HBV RT and/or HBV RT primer, can activate or inactivate the enzymatic nucleic acid domain of the nucleic acid sensor molecule, such that the activity of the nucleic acid sensor molecule is modulated in the presence of the target-signaling molecule. The nucleic acid sensor molecule can be designed to be active in the presence of the target molecule or alternately, can be designed to be inactive in the presence of the molecular target. For example, a nucleic acid sensor molecule is designed with a sensor domain having the sequence (UUCA)ₙ, where n is an integer from 1-10. In a non-limiting example, interaction of the HBV RT primer with the sensor domain of the nucleic acid sensor molecule can activate the enzymatic nucleic acid domain of the nucleic acid sensor molecule such that the sensor molecule catalyzes a reaction, for example cleavage of HBV RNA. In this example, the nucleic acid sensor molecule is activated in the presence of HBV RT or HBV RT primer, and can be used as a therapeutic to treat HBV infection. Alternately, the reaction can comprise cleavage or ligation of a labeled nucleic acid reporter molecule, providing a useful diagnostic reagent to detect the presence of HBV in a system.

Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., decoy nucleic acid molecules, aptamer nucleic acid molecules antisense nucleic acid molecules, enzymatic nucleic acid molecules) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., DNA oligonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table III outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for normal RNA including certain decoy nucleic acid molecules and enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table III outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides are synthesized by substituting a U for G₅ and a U for A₁₄ (numbering from Hertel, K. J., et al., 1992, Nucleic Acids Res., 20, 3252). Similarly, one or more nucleotide substitutions can be introduced in other nucleic acid decoy molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes can be purified by gel electrophoresis using general methods or can be purified by high pressure liquid chromatography (HPLC; see Wincott et al., supra, the totality of which is hereby incorporated herein by reference) and re-suspended in water.

The sequences of the decoy constructs that are chemically synthesized, useful in this study, are shown in Table I. The decoy construct sequences listed in Table I can be formed of ribonucleotides or other nucleotides or non-nucleotides. Such decoys are equivalent to the decoy sequences described specifically in the Table.

Optimizing Activity of the Nucleic Acid Molecule of the Invention.

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without modulating catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore, when designing nucleic acid molecules, the amount of these internucleotide linkages should be evaluated and minimized as necessary to lower toxicity, resulting in increased efficacy and higher specificity of these molecules.

Nucleic acid molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein)) have expanded the ability to modify nucleic acid molecules by introducing nucleotide modifications to enhance their nuclease stability, as described above.

In one embodiment, nucleic acid molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets. In another embodiment, nucleic acid molecules of the invention include one or more LNA “locked nucleic acid” nucleotides, such as a 2′, 4′-C mythylene bicyclo nucleotide (see for example Wengel et al., International PCT Publication No. WO 00/66604 and WO 99/14226).

In another embodiment, the invention features conjugates and/or complexes of nucleic acid molecules targeting HBV. Such conjugates and/or complexes can be used to facilitate delivery of molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example, 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules (e.g., decoy nucleic acid molecules) delivered exogenously optimally are stable within cells until reverse transcription of the pregenomic RNA has been modulated long enough to reduce the levels of HBV DNA. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, nucleic acid molecules having chemical modifications that maintain or enhance enzymatic activity are provided. Such nucleic acids are also generally more resistant to nucleases than unmodified nucleic acids. Thus, in vitro and/or in vivo the activity should not be significantly lowered. As exemplified herein, such nucleic acid molecules are useful in vitro and/or in vivo even if activity over all is reduced 10 fold (Burgin et al., 1996, Biochemistry, 35, 14090).

Use of the nucleic acid-based molecules of the invention can lead to better treatment of the disease progression by affording the possibility of combination therapies (e.g., multiple antisense, nucleic acid decoy, or nucleic acid aptamer molecules targeted to different genes; nucleic acid molecules coupled with known small molecule modulators ors; or intermittent treatment with combinations of molecules (including different motifs) and/or other chemical or biological molecules). The treatment of subjects with nucleic acid molecules can also include combinations of different types of nucleic acid molecules.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details, see Wincott et al., International PCT publication No. WO 97/26270, incorporated by reference herein).

In another embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. In one embodiment, the alkyl group has 1 to 12 carbons, for example, it is a lower alkyl of from 1 to 7 carbons, more specifically 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups that are unsaturated hydrocarbon groups containing at least one carbon-carbon double bond, including straight-chain, branched-chain, and cyclic groups. In one embodiment, the alkenyl group has 1 to 12 carbons. For example, it can be a lower alkenyl of from 1 to 7 carbons, more specifically 1 to 4 carbons. The alkenyl group can be substituted or unsubstituted. When substituted, the substituted group(s) can be, hydroxyl, cyano, alkoxy, ═O, ═S, NO₂, halogen, N(CH₃)₂, amino, or SH. The term “alkyl” also includes alkynyl groups that have an unsaturated hydrocarbon group containing at least one carbon-carbon triple bond, including straight-chain, branched-chain, and cyclic groups. In one emobodiment, the alkynyl group has 1 to 12 carbons. For example, it can be a lower alkynyl of from 1 to 7 carbons, more specifically 1 to 4 carbons. The alkynyl group can be substituted or unsubstituted. When substituted, the substituted group(s) can be, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of a nucleic acid decoy molecule and/or in the substrate-binding regions of the nucleic acid molecule.

In one embodiment, the invention features modified nucleic acids, for example decoys, with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which may be modified or unmodified. Such modified groups are described, for example, in Eckstein et al., U.S. Pat. No. 5,672,695 and Matulic-Adamic et al., WO 98/28317, which are both incorporated by reference in their entireties.

Various modifications to nucleic acid (e.g., decoy, aptamer, antisense and ribozyme) structure can be made to enhance the utility of these molecules. Such modifications can enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Sullivan et al., PCT WO 94/02595, further describes the general methods for delivery of enzymatic nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, ameliorate or treat (alleviate a symptom to some extent, preferably all of the symptoms) of a disease state in a subject.

Thus, the invention features a composition comprising one or more nucleic acid(s) of the invention in an pharmaceutically acceptable carrier, such as a stabilizer, buffer, and the like. The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

In one embodiment, a pharmaceutically acceptable composition or formulation refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective amount or dose is that dose required to prevent, inhibit the occurrence of, ameliorate or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example, sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication may increase the beneficial effects while reducing the presence of side effects.

In one embodiment, the invention compositions suitable for administering nucleic acid molecules of the invention to specific cell types, such as hepatocytes. For example, the asialoglycoprotein receptor (ASGPr) (Wu and Wu, 1987, J Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J Biol. Chem., 269, 25856; all of these references are hereby incorporated in their totality by reference herein).

In another aspect of the invention, nucleic acid molecules of the present invention are preferably expressed from transcription units (see, for example, Couture et al., 1996, TIG., 12, 510) inserted into DNA or RNA vectors. The recombinant vectors are preferably DNA plasmids or viral vectors. Nucleic acid expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the subject followed by reintroduction into the subject, or by any other means that would allow for introduction into the desired target cell (for a review see Couture et al., 1996, TIG., 12, 510).

In one aspect, the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention, in a manner that allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of nucleic acid decoy molecules that target HBV reverse transcriptase.

### Example 1

### Modulation of HBV Reverse Transcriptase

The HBV reverse transcriptase (pol) binds to the 5′ stem-loop structure in the HBV pregenomic RNA and synthesizes a four-nucleotide primer from the template UUCA. The reverse transcriptase then translocates to the 3′ end of the pregenomic RNA where the primer binds to the UUCA sequence within the DR1 element and begins first-strand synthesis of HBV DNA. A number of short oligos, ranging in size from 4 to 16-mers, were designed to act as competitive inhibitors of the HBV reverse transcriptase primer, either by blocking the primer binding sites on the HBV RNA or by acting as a decoy.

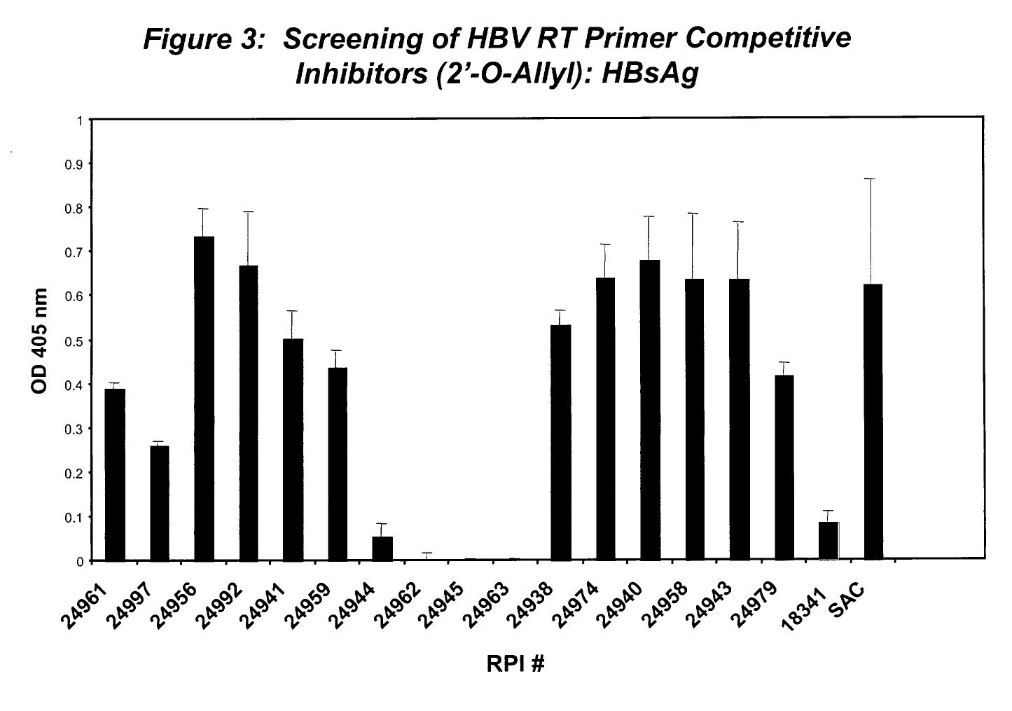

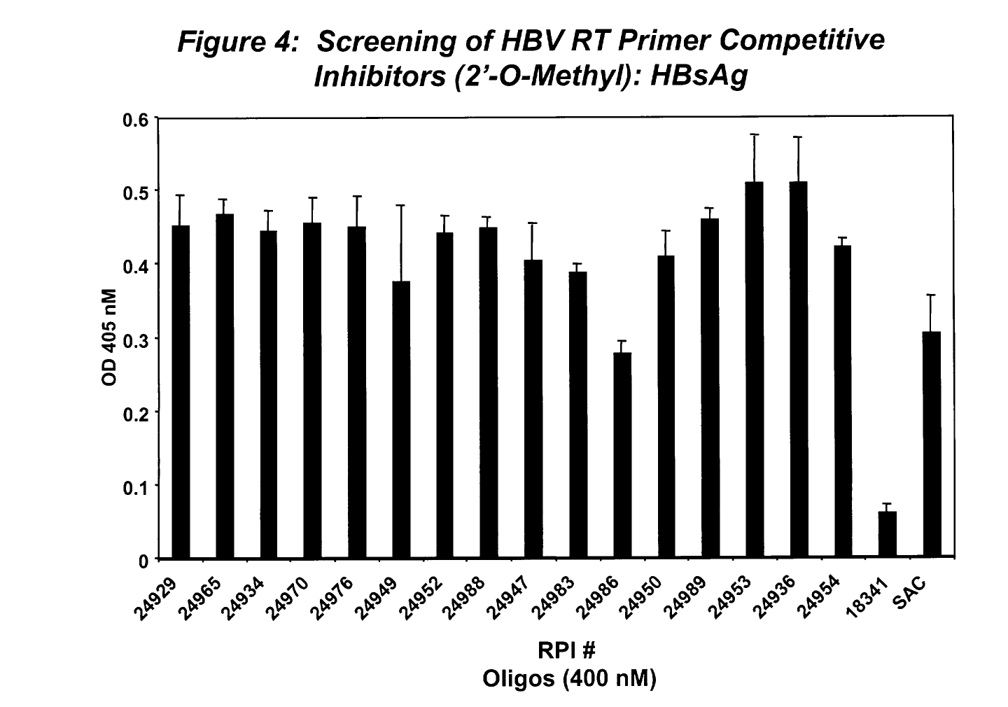

The oligonucleotides and controls were synthesized in all 2′-O-methyl and 2′-O-allyl versions (Table II). The inverse sequence of all oligos were generated to serve as controls. Primary screening of the competitive inhibitors was completed in the HBsAg transfection/ELISA system, in which the oligo is co-transfeceted with a HBV cDNA vector into Hep G2 cells. Following 4 days of incubation, the levels of HBsAg secreted into the cell culture media were determined by ELISA. Screening of the 2′-O-allyl versions revealed that two of the decoy oligos (RPI.24944 and RPI.24945), consisting of 3× or 4×repeats of the RT primer binding site UUCA, along with the matched inverse controls, displayed considerable activity by decreasing HBsAg levels (FIG. 3). This dramatic decrease in HBsAg levels is not due to cellular toxicity, because a MTS assay showed no difference in proliferation between any of the treated cells. A follow up experiment with a 5×UUCA repeat, the inverse sequence control, and a matched scrambled control, showed that all three oligos decreased HBsAg levels without cellular toxicity. Screening of the 2′-O-methyl versions of the oligos showed no activity from the 3× and 4×UUCA repeat (FIG. 4), suggesting that the anti-HBV effect could be related to the 2′-O-allyl chemistry rather than to sequence specificity.

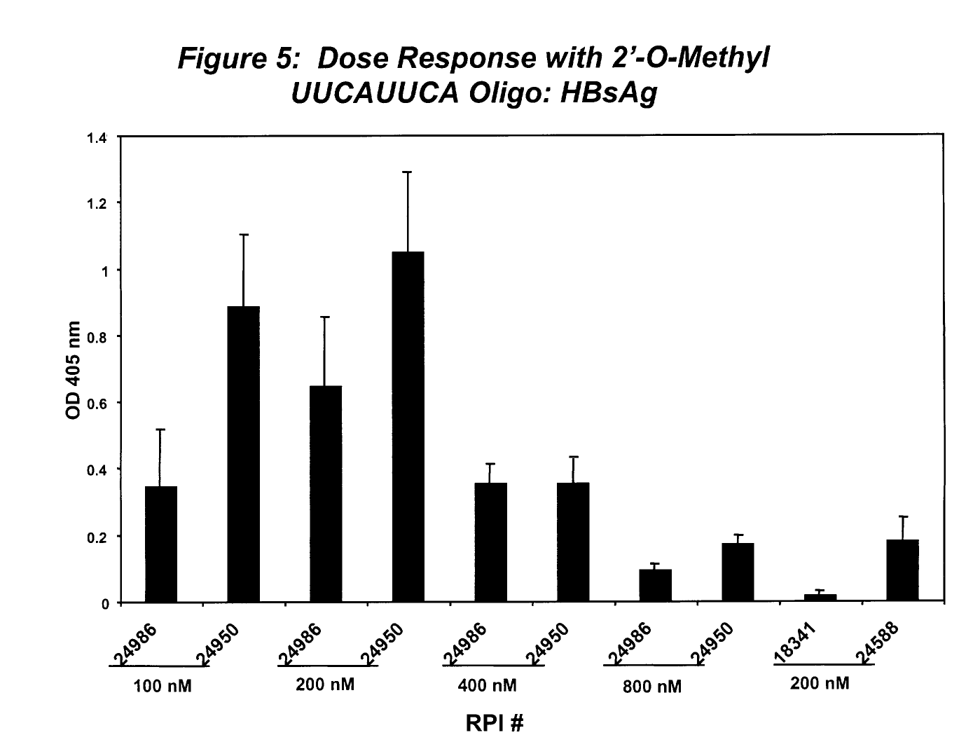

Screening of the 2′-O-methyl oligos did show that the 2′-O-methyl 2×UUCA repeat, RPI.24986, displayed activity in decreasing HBsAg levels as compared to the inverse control, RPI.24950. A dose response experiment showed that at the lower concentrations of 100 and 200 nM, RPI.24986 showed greater activity in decreasing HbsAg levels as compared to the inverse control RPI.24950 (FIG. 5).

### Example 2

### Modulation of HBV Transcription via Oligonucleotides Targeting the Enchancer I Core Region of HBV DNA

In an effort to block HBV replication, oligonucleotides were designed to bind to two liver-specific factor binding sites in the Enhancer I core region of HBV genomic DNA. Hepatocyte Nuclear Factor 3 (HNF3) and Hepatocyte Nuclear Factor 4 (HNF4) bind to sites in the core region, with the HNF3 site being 5′ to the HNF4 site. The HNF3 and HNF4 sites overlap or are adjacent to binding sites for a number of more ubiquitous factors, and are termed nuclear receptor response elements (NRRE). These elements are critical in regulating HBV transcription and replication in infected hepatocytes, with mutations in the HNF3 and HNF4 binding sites having been demonstrated to greatly reduce the levels of HBV replication (Bock et al., 2000, J. Virology, 74, 2193)

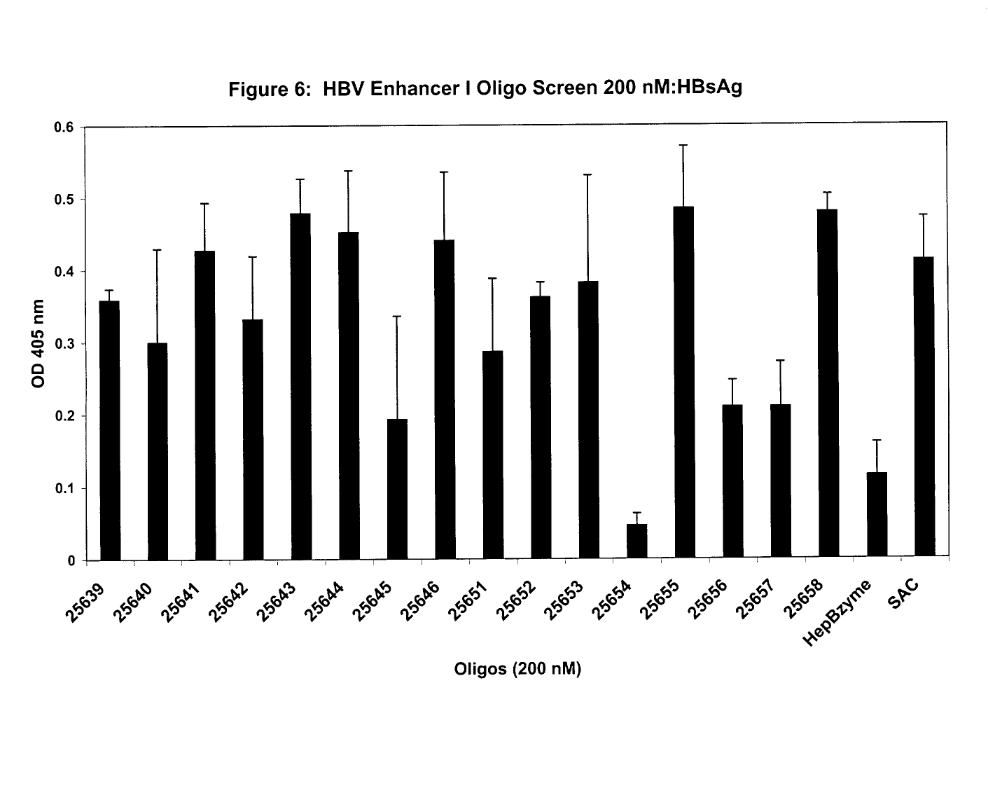

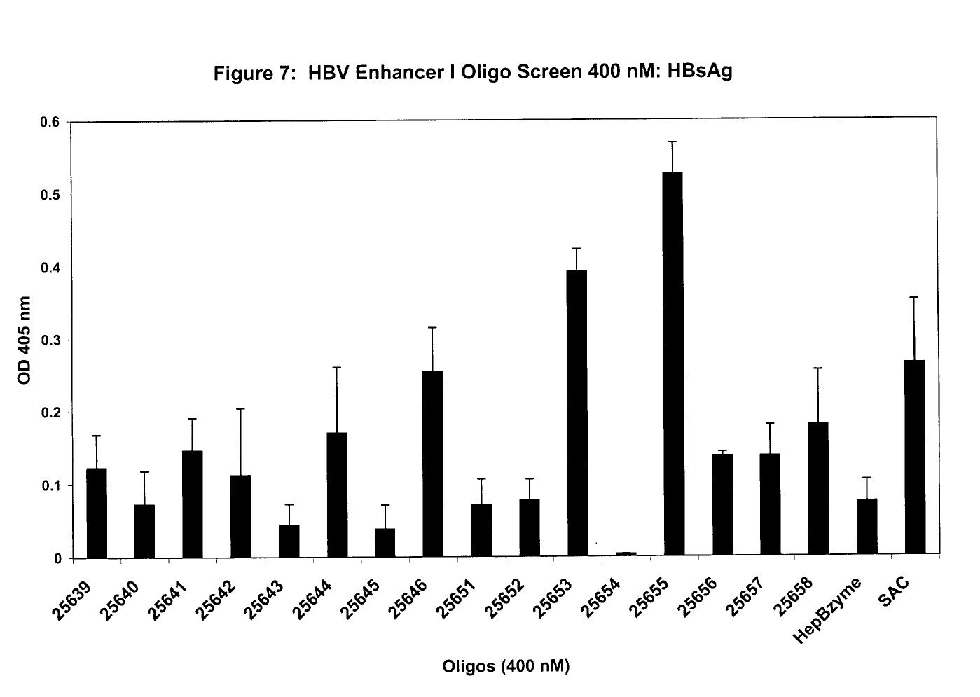

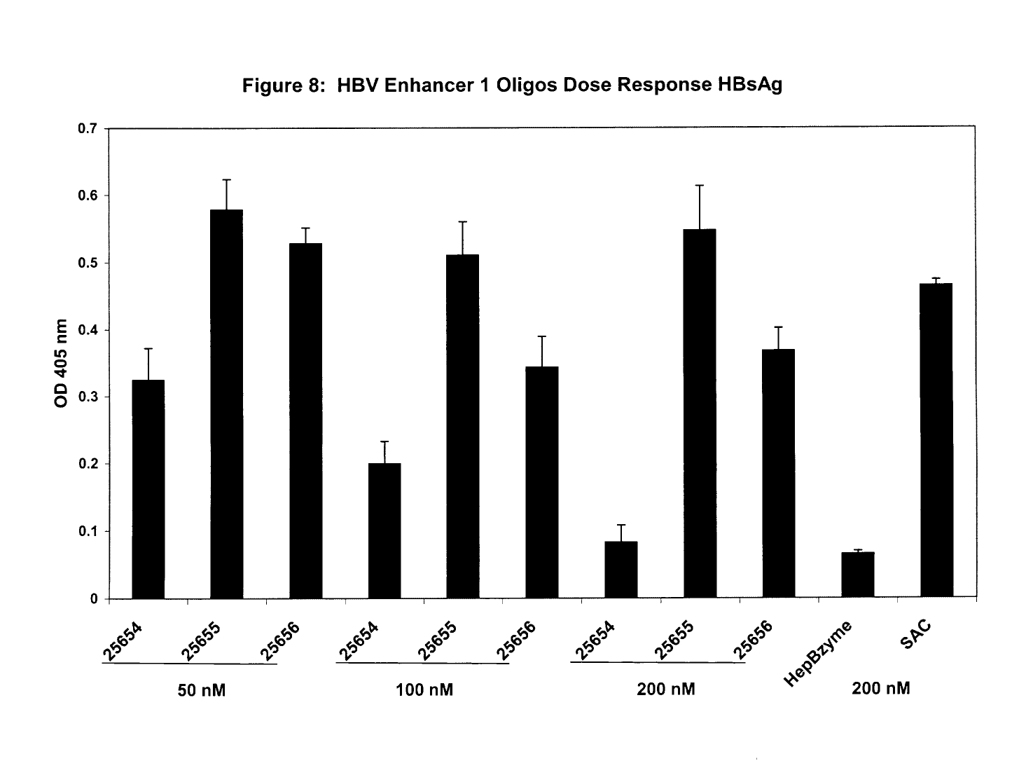

Oligonucleotides (Table II) were designed to bind to either the positive or negative strands of the HNF3 or HNF4 binding sites. Scrambled controls were made to match each oligo. Each oligo was synthesized in all 2′-O-methyl/all phosphorothioate, or all 2′-O-allyl/all phosphorothioate chemistries. The initial screening of the oligos was done in the HBsAg transfection/ELISA system in Hep G2 cells as described in Example 1. RPI.25654, which targets the negative strand of the HNF4 binding site, shows greater activity in reducing HBsAg levels as compared to RPI.25655, which targets the HNF4 site positive strand, and the scrambled control RPI.25656. This result was observed at both 200 and 400 nM (FIGS. 6 and 7). In a follow-up study, RPI.25654 reduced HBsAg levels in a dose-dependent manner, from 50-200 nM (FIG. 8).

### Example 3

### Transfection of HepG2 Cells With psHBV-1 and Nucleic Acid

The human hepatocellular carcinoma cell line Hep G2 was grown in Dulbecco's modified Eagle media supplemented with 10% fetal calf serum, 2 mM glutamine, 0.1 mM nonessential amino acids, 1 mM sodium pyruvate, 25 mM Hepes, 100 units penicillin, and 100 μg/ml streptomycin. To generate a replication competent cDNA, prior to transfection the HBV genomic sequences are excised from the bacterial plasmid sequence contained in the psHBV-1 vector This was done with an EcoRI and Hind III restriction digest. Following completion of the digest, a ligation was performed under dilute conditions (20 μg/ml) to favor intermolecular ligation. The total ligation mixture was then concentrated using Qiagen spin columns. One skilled in the art would realize that other methods can be used to generate a replication competent cDNA

Secreted alkaline phosphatase (SEAP) was used to normalize the HBsAg levels to control for transfection variability. The pSEAP2-TK control vector was constructed by ligating a Bgl II-Hind III fragment of the pRL-TK vector (Promega), containing the herpes simplex virus thymidine kinase promoter region, into Bgl II/Hind III digested pSEAP2-Basic (Clontech). Hep G2 cells were plated (3×10⁴ cells/well) in 96-well microtiter plates and incubated overnight. A lipid/DNA/nucleic acid complex was formed containing (at final concentrations) cationic lipid (15 μg/ml), prepared psHBV-1 (4.5 μg/ml), pSEAP2-TK (0.5 μg/ml), and nucleic acid (100 μM). Following a 15 min. incubation at 37° C., the complexes were added to the plated Hep G2 cells. Media was removed from the cells 96 hour post-transfection for HBsAg and SEAP analysis.

Transfection of the human hepatocellular carcinoma cell line, Hep G2, with replication competent HBV DNA results in the expression of HBV proteins and the production of virions.

### Example 4

### Analysis of HBsAg and SEAP Levels Following Nucleic Acid Treatment

Immulon 4 (Dynax) microtiter wells were coated overnight at 4° C. with anti-HBsAg Mab (Biostride B88-95-31ad,ay) at 1 μg/ml in Carbonate Buffer (Na2CO3 15 mM, NaHCO3 35 mM, pH 9.5). The wells were then washed 4× with PBST (PBS, 0.05% Tween® 20) and blocked for 1 hr at 37° C. with PBST, 1% BSA. Following washing as above, the wells were dried at 37° C. for 30 minutes. Biotinylated goat anti-HBsAg (Accurate YVS 1807) was diluted 1:1000 in PBST and incubated in the wells for 1 hour at 37° C. The wells were washed 4× with PBST. Streptavidin/Alkaline Phosphatase Conjugate (Pierce 21324) was diluted to 250 ng/ml in PBST, and incubated in the wells for 1 hour at 37° C. After washing as above, p-nitrophenyl phosphate substrate (Pierce 37620) was added to the wells, which were then incubated for 1 hour at 37° C. The optical density at 405 nm was then determined. SEAP levels were assayed using the Great EscAPe® Detection Kit (Clontech K2041-1), as per the manufacturers instructions.

### Example 5

### Analysis of HBV DNA Expression a HepG2.2.15 Murine Model

The development of new antiviral agents for the treatment of chronic Hepatitis B has been aided by the use of animal models that are permissive to replication of related Hepadnaviridae such as Woodchuck Hepatitis Virus (WHV) and Duck Hepatitis Virus (DHV). In addition, the use of transgenic mice has also been employed. The human hepatoblastoma cell line, HepG2.2.15, implanted as a subcutaneous (SC) tumor, can be used to produce Hepatitis B viremia in mice. This model is useful for evaluating new HBV therapies. Mice bearing HepG2.2.15 SC tumors show HBV viremia. HBV DNA can be detected in serum beginning on Day 35. Maximum serum viral levels reach 1.9×10⁵ copies/mL by day 49. A study also determined that the minimum tumor volume associated with viremia was 300 mm³. Therefore, the HepG2.2.15 cell line grown as a SC tumor produces a useful model of HBV viremia in mice. This new model can be used to evaluate new therapeutic regimens for chronic Hepatitis B.

HepG2.2.15 tumor cells contain a slightly truncated version of viral HBV DNA and sheds HBV particles. The purpose of this study was to identify what time period viral particles are shed from the tumor. Serum was analyzed for presence of HBV DNA over a time course after HepG2.2.15 tumor inoculation in Athymic Ncr nu/nu mice. HepG2.2.15 cells were carried and expanded in DMEM/10% FBS/2.4% HEPES/1% NEAA/1% Glutamine/1% Sodium Pyruvate media. Cells were resuspended in Delbecco's PBS with calcium/magnesium for injection. One hundred microliters of the tumor cell suspension (at a concentration of 1×10⁸ cells/mL) were injected subcutaneously in the flank of NCR nu/nu female mice with a 23 μl needle and 1 cc syringe, thereby giving each mouse 1×10⁷ cells. Tumors were allowed to grow for a period of up to 49 days post tumor cell inoculation. Serum was sampled for analysis on days 1, 7, 14, 35, 42 and 49 post tumor inoculation. Length and width measurements from each tumor were obtained three times per week using a Jamison microcaliper. Tumor volumes were calculated from tumor length/width measurements (tumor volume=0.5[a(b)²] where a=longest axis of the tumor and b=shortest axis of the tumor). Serum was analyzed for the presence of HBV DNA by the Roche Amplicor HBV moniter TM DNA assay.

Cell Culture Models

As previously mentioned, HBV does not infect cells in culture. However, transfection of HBV DNA (either as a head-to-tail dimer or as an “overlength” genome of >100%) into HuH7 or Hep G2 hepatocytes results in viral gene expression and production of HBV virions released into the media. Thus, HBV replication competent DNA are co-transfected with ribozymes in cell culture. Such an approach has been used to report intracellular ribozyme activity against HBV (zu Putlitz, et al., 1999, J. Virol., 73, 5381-5387, and Kim et al., 1999, Biochem. Biophys. Res. Commun., 257, 759-765). In addition, stable hepatocyte cell lines have been generated that express HBV. In these cells, only ribozyme need be delivered; however, performance of a delivery screen is required. Intracellular HBV gene expression can be assayed by a Taqman® assay for HBV RNA or by ELISA for HBV protein. Extracellular virus can be assayed by PCR for DNA or ELISA for protein. Antibodies are commercially available for HBV surface antigen and core protein. A secreted alkaline phosphatase expression plasmid can be used to normalize for differences in transfection efficiency and sample recovery.

Animal Models

There are several small animal models to study HBV replication. One is the transplantation of HBV-infected liver tissue into irradiated mice. Viremia (as evidenced by measuring HBV DNA by PCR) is first detected 8 days after transplantation and peaks between 18-25 days (Ilan et al., 1999, Hepatology, 29, 553-562).

Transgenic mice that express HBV have also been used as a model to evaluate potential anti-virals. HBV DNA is detectable in both liver and serum (Guidotti et al., 1995, J. Virology, 69, 10, 6158-6169; Morrey et al., 1999, Antiviral Res., 42, 97-108).

An additional model is to establish subcutaneous tumors in nude mice with Hep G2 cells transfected with HBV. Tumors develop in about 2 weeks after inoculation and express HBV surface and core antigens. HBV DNA and surface antigen is also detected in the circulation of tumor-bearing mice (Yao et al., 1996, J. Viral Hepat., 3, 19-22).

In one embodiment, the invention features a mouse, for example a male or female mouse, implanted with HepG2.2.15 cells, wherein the mouse is susceptible to HBV infection and capable of sustaining HBV DNA expression. One embodiment of the invention provides a mouse implanted with HepG2.2.15 cells, wherein said mouse sustains the propagation of HEPG2.2.15 cells and HBV production (see Macejak, U.S. Provisional Patent Application No. 60/296,876).

Woodchuck hepatitis virus (WHV) is closely related to HBV in its virus structure, genetic organization, and mechanism of replication. As with HBV in humans, persistent WHV infection is common in natural woodchuck populations and is associated with chronic hepatitis and hepatocellular carcinoma (HCC). Experimental studies have established that WHV causes HCC in woodchucks and woodchucks chronically infected with WHV have been used as a model to lest a number of anti-viral agents. For example, the nucleoside analogue 3T3 was observed to cause dose dependent reduction in virus (50% reduction after two daily treatments at the highest dose) (Hurwitz et al., 1998. Antimicrob. Agents Chemother., 42, 2804-2809).

Indications

Particular degenerative and disease states that can be associated with HBV expression modulation include, but are not limited to, HBV infection, hepatitis, cancer, tumorigenesis, cirrhosis, liver failure and others.

The present body of knowledge in HBV research indicates the need for methods to assay HBV activity and for compounds that can regulate HBV expression for research, diagnostic, and therapeutic use.

Lamivudine (3TC®), L-FMAU, adefovir dipivoxil, type 1 Interferon (e.g, interferon alpha, interferon beta, consensus interferon, polyethylene glycol interferon, polyethylene glycol interferon alpha 2a, polyethylene glycol interferon 2b, and polyethylene glycol consensus interferon), therapeutic vaccines, steriods, and 2′-5′ Oligoadenylates are non-limiting examples of pharmaceutical agents that can be combined with or used in conjunction with the nucleic acid molecules of the instant invention. Those skilled in the art will recognize that other drugs or other therapies can similarly and readily be combined with the nucleic acid molecules of the instant invention and are, therefore, within the scope of the instant invention.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations that are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I

HBV RT primer Decoy sequences

| | | |
|---|---|---|
| | | Seq ID |
| Length | Decoy Sequence | No. |

| | | |
|---|---|---|
| 4 | AUUC | 1 |
| 4 | CAUU | 2 |
| 4 | UCAU | 3 |
| 4 | UUCA | 4 |
| 5 | AUUCA | 5 |
| 5 | CAUUC | 6 |
| 5 | UCAUU | 7 |
| 5 | UUCAU | 8 |
| 6 | AUUCAU | 9 |
| 6 | CAUUCA | 10 |
| 6 | UCAUUC | 11 |
| 6 | UUCAUU | 12 |
| 7 | AUUCAUU | 13 |
| 7 | CAUUCAU | 14 |
| 7 | UCAUUCA | 15 |
| 7 | UUCAUUC | 16 |
| 8 | AUUCAUUC | 17 |
| 8 | CAUUCAUU | 18 |
| 8 | UCAUUCAU | 19 |
| 8 | UUCAUUCA | 20 |
| 9 | AUUCAUUCA | 21 |
| 9 | CAUUCAUUC | 22 |
| 9 | UCAUUCAUU | 23 |
| 9 | UUCAUUCAU | 24 |
| 10 | AUUCAUUCAU | 25 |
| 10 | CAUUCAUUCA | 26 |
| 10 | UCAUUCAUUC | 27 |
| 10 | UUCAUUCAUU | 28 |
| 11 | AUUCAUUCAUU | 29 |
| 11 | CAUUCAUUCAU | 30 |
| 11 | UCAUUCAUUCA | 31 |
| 11 | UUCAUUCAUUC | 32 |
| 12 | AUUCAUUCAUUC | 33 |
| 12 | CAUUCAUUCAUU | 34 |
| 12 | UCAUUCAUUCAU | 35 |
| 12 | UUCAUUCAUUCA | 36 |
| 13 | AUUCAUUCAUUCA | 37 |
| 13 | CAUUCAUUCAUUC | 38 |
| 13 | UCAUUCAUUCAUU | 39 |
| 13 | UUCAUUCAUUCAU | 40 |
| 14 | AUUCAUUCAUUCAU | 41 |
| 14 | CAUUCAUUCAUUCA | 42 |
| 14 | UCAUUCAUUCAUUC | 43 |
| 14 | UUCAUUCAUUCAUU | 44 |
| 15 | AUUCAUUCAUUCAUU | 45 |
| 15 | CAUUCAUUCAUUCAU | 46 |
| 15 | UCAUUCAUUCAUUCA | 47 |
| 15 | UUCAUUCAUUCAUUC | 48 |
| 16 | AUUCAUUCAUUCAUUC | 49 |
| 16 | CAUUCAUUCAUUCAUU | 50 |
| 16 | UCAUUCAUUCAUUCAU | 52 |
| 16 | UUCAUUCAUUCAUUCA | 52 |
| 17 | AUUCAUUCAUUCAUUCA | 53 |
| 17 | CAUUCAUUCAUUCAUUC | 54 |
| 17 | UCAUUCAUUCAUUCAUU | 55 |
| 17 | UUCAUUCAUUCAUUCAU | 56 |
| 18 | AUUCAUUCAUUCAUUCAU | 57 |
| 18 | CAUUCAUUCAUUCAUUCA | 58 |
| 18 | UCAUUCAUUCAUUCAUUC | 59 |
| 18 | UUCAUUCAUUCAUUCAUU | 60 |
| 19 | AUUCAUUCAUUCAUUCAUU | 61 |
| 19 | CAUUCAUUCAUUCAUUCAU | 62 |
| 19 | UCAUUCAUUCAUUCAUUCA | 63 |
| 19 | UUCAUUCAUUCAUUCAUUC | 64 |
| 20 | AUUCAUUCAUUCAUUCAUUC | 65 |
| 20 | CAUUCAUUCAUUCAUUCAUU | 66 |
| 20 | UCAUUCAUUCAUUCAUUCAU | 67 |
| 20 | UUCAUUCAUUCAUUCAUUCA | 68 |
| 21 | AUUCAUUCAUUCAUUCAUUCA | 69 |
| 21 | CAUUCAUUCAUUCAUUCAUUC | 70 |
| 21 | UCAUUCAUUCAUUCAUUCAUU | 71 |
| 21 | UUCAUUCAUUCAUUCAUUCAU | 72 |
| 22 | CAUUCAUUCAUUCAUUCAUUCA | 73 |
| 22 | UCAUUCAUUCAUUCAUUCAUUC | 74 |
| 22 | UUCAUUCAUUCAUUCAUUCAUU | 75 |
| 23 | UCAUUCAUUCAUUCAUUCAUUCA | 76 |
| 23 | UUCAUUCAUUCAUUCAUUCAUUC | 77 |
| 24 | UUCAUUCAUUCAUUCAUUCAUUCA | 78 |

TABLE II

Synthetic Nucleic acid molecules

| | | | |
|---|---|---|---|
| RPI# | Alias | Sequence | SeqID |

| | | | |
|---|---|---|---|
| 24961 | HBV DR1 2′Oallyl P = S | gₛcₛaₛgₛaₛgₛgₛuₛgₛaₛaₛB | 79 |
| 24997 | HBV DR1 2′Oallyl P = S control | aₛaₛgₛuₛgₛgₛaₛgₛaₛcₛgₛB | 80 |
| 24956 | HBV 1866-1869 1 × 2′Oallyl P = S | uₛuₛcₛaₛB | 81 |
| 24992 | HBV 1866-1869 1 × 2′Oallyl P = S control | aₛcₛuₛuₛB | 82 |
| 24941 | HBV 1866-1869 2 × 2′Oallyl P = S | uₛuₛcₛaₛuₛuₛcₛaₛB | 83 |
| 24959 | HBV 1866-1869 2 × 2′Oallyl P = S control | aₛcₛuₛuₛaₛcₛuₛuₛB | 84 |
| 24944 | HBV 1866-1869 3 × 2′Oallyl P =S | uₛuₛcₛaₛuₛuₛcₛaₛuₛuₛcₛaₛB | 85 |
| 24962 | HBV 1866-1869 3 × 2′Oallyl P = S control | aₛcₛuₛuₛaₛcₛuₛuₛaₛcₛuₛuₛB | 86 |
| 24945 | HBV 1866-1869 4 × 2′Oallyl P = S | uₛuₛcₛaₛuₛuₛcₛaₛuₛuₛcₛaₛuₛuₛcₛaₛB | 87 |
| 24963 | HBV 1866-1869 4 × 2′Oallyl P = S control | aₛcₛuₛuₛaₛcₛuₛuₛaₛcₛuₛuₛaₛcₛuₛuₛB | 88 |
| 24938 | HBV 1866-1869 2′Oallyl P = S | uₛgₛaₛaₛB | 89 |
| 24974 | HBV 1866-1869 2′Oallyl P = S control | aₛaₛgₛuₛB | 90 |
| 24940 | HBV 1866-1872 2′Oallyl P = S | gₛcₛuₛuₛgₛaₛaₛB | 91 |
| 24958 | HBV 1866-1872 2′Oallyl P = S control | aₛaₛgₛuₛuₛcₛgₛB | 92 |
| 24943 | HBV 1866-1876 2′Oallyl P = S | gₛgₛaₛgₛgₛcₛuₛuₛgₛaₛaB | 93 |
| 24979 | HBV 1866-1876 2′Oallyl P = S control | aₛaₛgₛuₛuₛcₛgₛgₛaₛgₛgₛB | 94 |
| 18341 | HBV-273 UH.Rz-7 allyl stab1 | gₛaₛaₛaₛauu cUGAuGaggccguuaggccGaa | 95 |
| | | Agagaag B | |
| 24588 | HBV-273 UH.Rz-7 allyl stab1 inact3 scram1 | aₛaₛuₛgₛagg cUAGuGacgccguuaggcgGaa | 96 |
| | (GUUA SAC) | Aaaugaa B | |
| 24929 | HBV 1866-1969 2′Omethyl | ugaaB | 97 |
| 24965 | HBV 1866-1969 2′Omethyl control | aaguB | 98 |
| 24934 | HBV 1866-1876 2′Omethyl | ggaggcuugaaB | 99 |
| 24970 | HBV 1866-1876 2′Omethyl control | aaguucggaggB | 100 |
| 24976 | HBV 1866-1872 2′Omethyl | gcuugaaB | 101 |
| 24949 | HBV 1866-1872 2′Omethyl control | aaguucgB | 102 |
| 24952 | HBV DR1 2′Omethyl | gcagaggugaaB | 103 |
| 24988 | HBV DR1 2′Omethyl control | aaguggagacgB | 104 |
| 24947 | HBV 1866-1869 1 × 2′Omethyl | uucaB | 105 |
| 24983 | HBV 1866-1869 1 × 2′Omethyl control | acuuB | 106 |
| 24986 | HBV 1866-1869 2 × 2′Omethyl | uucauucaB | 107 |
| 24950 | HBV 1866-1869 2 × 2′Omethyl control | acuuacuuB | 108 |
| 24989 | HBV 1866-1869 3 × 2′Omethyl | uucauucauucaB | 109 |
| 24953 | HBV 1866-1869 3 × 2′Omethyl control | acuuacuuacuuB | 110 |
| 24936 | HBV 1866-1869 4 × 2′Omethyl | uucauucauucauucaB | 111 |
| 24954 | HBV 1866-1869 4 × 2′Omethyl control | acuuacuuacuuacuuB | 112 |
| 25639 | HBV 5′ Enl pos OMe P = S | B uₛuₛuₛcₛuₛaₛaₛgₛuₛaₛaₛaₛcₛaₛgₛu B | 113 |
| 25640 | HBV 5′ Enl neg OMe P = S | B aₛcₛuₛgₛuₛuₛuₛaₛcₛuₛuₛaₛgₛaₛaₛa B | 114 |
| 25641 | HBV 5′ Enl sc OMe P = S | B aₛaₛgₛuₛaₛaₛcₛuₛcₛuₛaₛuₛgₛuₛuₛa B | 115 |
| 25642 | HBV 3′ Enl pos OMe P = S | B uₛaₛcₛaₛuₛgₛaₛaₛcₛcₛuₛuₛuₛaₛcₛcₛcₛc B | 116 |
| 25643 | HBV 3′ Enl neg OMe P = S | B gₛgₛgₛuₛaₛaₛaₛgₛgₛuₛuₛcₛaₛuₛgₛuₛa B | 117 |
| 25644 | HBV 3′ Enl pos sc OMe P = S | B aₛcₛcₛuₛaₛuₛcₛgₛcₛcₛuₛaₛcₛuₛcₛuₛaₛa B | 118 |
| 25645 | HBV 5′ Enl neg sc OMe P = S | B uₛgₛaₛuₛaₛgₛcₛgₛgₛaₛuₛgₛaₛgₛaₛuₛu B | 119 |
| 25646 | HBV DR1 pos OMe P = S | B uₛuₛcₛaₛcₛcₛuₛcₛuₛgₛc B | 120 |
| 25651 | HBV 5′ Enl pos Oallyl P = S | B uₛuₛuₛcₛuₛaₛaₛgₛuₛaₛaₛaₛcₛaₛgₛu B | 121 |
| 25652 | HBV 5′ Enl neg Oallyl P = S | B aₛcₛuₛgₛuₛuₛuₛaₛcₛuₛuₛaₛgₛaₛaₛa B | 122 |
| 25653 | HBV 5′ Enl sc Oallyl P = S | B aₛaₛgₛuₛaₛaₛcₛuₛcₛuₛaₛuₛgₛuₛuₛa B | 123 |
| 25654 | HBV 3′ Enl pos Oallyl P = S | B uₛaₛcₛaₛuₛgₛaₛaₛcₛcₛuₛuₛuₛaₛcₛcₛcₛc B | 124 |
| 25655 | HBV 3′ Enl neg Oallyl P = S | B gₛgₛgₛuₛaₛaₛaₛgₛgₛuₛuₛcₛaₛuₛgₛuₛa B | 125 |
| 25656 | HBV 3′ Enl pos sc Oallyl P = S | B aₛcₛcₛuₛaₛuₛcₛgₛcₛcₛuₛaₛcₛuₛcₛuₛaₛa B | 126 |
| 25657 | HBV 5′ Enl neg sc Oallyl P = S | B uₛgₛaₛuₛaₛgₛcₛgₛgₛaₛuₛgₛaₛgₛaₛuₛu B | 127 |
| 25658 | HBV DR1 pos Oallyl P = S | B uₛuₛcₛaₛcₛcₛuₛcₛuₛgₛc B | 128 |

a, g, c, u = all 2′-O-allyl
a, g, c, u = 2′-O-methyl
U = 2′-C-allyl Uridine
S = phosphorothioate
B = inverted deoxyabasic

TABLE III
A. 2.5 μmol Synthesis Cycle ABI 394 Instrument

| | | | | | |
|---|---|---|---|---|---|
| | | | | Wait Time* 2′-O- | |
| Reagent | Equivalents | Amount | Wait Time* DNA | methyl | Wait Time*RNA |

| | | | | | | | | |
|---|---|---|---|---|---|---|---|---|
| Phosphoramidites | 6.5 | 163 | μL | 45 sec | 2.5 | min | 7.5 | min |
| S-Ethyl Tetrazole | 23.8 | 238 | μL | 45 sec | 2.5 | min | 7.5 | min |
| Acetic Anhydride | 100 | 233 | μL | 5 sec | 5 | sec | 5 | sec |
| N-Methyl | 186 | 233 | μL | 5 sec | 5 | sec | 5 | sec |
| Imidazole | | | | | | | | |
| TCA | 176 | 2.3 | mL | 21 sec | 21 | sec | 21 | sec |
| Iodine | 11.2 | 1.7 | mL | 45 sec | 45 | sec | 45 | sec |
| Beaucage | 12.9 | 645 | μL | 100 sec | 300 | sec | 300 | sec |
| Acetonitrile | NA | 6.67 | mL | NA | NA | | NA | |

B. 0.2 μmol Synthesis Cycle ABI 394 Instrument

| | | | | | |
|---|---|---|---|---|---|
| | | | | Wait Time 2′-O- | |
| Reagent | Equivalents | Amount | Wait Time* DNA | methyl | Wait Time*RNA |

| | | | | | | | |
|---|---|---|---|---|---|---|---|
| Phosphoramidites | 15 | 31 | μL | 45 sec | 233 | sec | 465 sec |
| S-Ethyl Tetrazole | 38.7 | 31 | μL | 45 sec | 233 | min | 465 sec |
| Acetic Anhydride | 655 | 124 | μL | 5 sec | 5 | sec | 5 sec |
| N-Methyl | 1245 | 124 | μL | 5 sec | 5 | sec | 5 sec |
| Imidazole | | | | | | | |
| TCA | 700 | 732 | μL | 10 sec | 10 | sec | 10 sec |
| Iodine | 20.6 | 244 | μL | 15 sec | 15 | sec | 15 sec |
| Beaucage | 7.7 | 232 | μL | 100 sec | 300 | sec | 300 sec |
| Acetonitrile | NA | 2.64 | mL | NA | NA | | NA |

C. 0.2 μmol Synthesis Cycle 96 well Instrument

| | Equivalents:DNA/ | Amount: DNA/2′-O- | | | Wait Time* 2′-O- | |
|---|---|---|---|---|---|---|
| Reagent | 2′-O-methyl/Ribo | methyl/Ribo | | Wait Time* DNA | methyl | Wait Time* Ribo |
| Phosphoramidites | 22/33/66 | 40/60/120 | μL | 60 sec | 180 sec | 360 sec |
| S-Ethyl Tetrazole | 70/105/210 | 40/60/120 | μL | 60 sec | 180 min | 360 sec |
| Acetic Anhydride | 265/265/265 | 50/50/50 | μL | 10 sec | 10 sec | 10 sec |
| N-Methyl | 502/502/502 | 50/50/50 | μL | 10 sec | 10 sec | 10 sec |
| Imidazole | | | | | | |
| TCA | 238/475/475 | 250/500/500 | μL | 15 sec | 15 sec | 15 sec |
| Iodine | 6.8/6.8/6.8 | 80/80/80 | μL | 30 sec | 30 sec | 30 sec |
| Beaucage | 34/51/51 | 80/120/120 | | 100 sec | 200 sec | 200 sec |
| Acetonitrile | NA | 1150/1150/1150 | μL | NA | NA | NA |

*Wait time does not include contact time during delivery.

What we claim is:

1. A nucleic acid molecule that specifically binds the hepatitis B virus (HBV) reverse transcriptase primer, wherein said nucleic acid molecule comprises the sequence (UUCA)n, wherein n is an integer from 1 to 10.

2. A nucleic acid molecule that specifically binds the hepatitis B virus (HBV) reverse transcriptase primer, wherein said nucleic acid molecule comprises any of SEQ ID NOs: 1-79, 81, 83, 85, 87, 89, 91, 93, 97, 99, 101, 103, 105, 107, 109, and 111.

3. A nucleic acid molecule that specifically binds to the Enhancer I sequence of HBV DNA.

4. A nucleic acid molecule that specifically binds to the Enhancer I region of HBV DNA, wherein said nucleic acid molecule comprises any of SEQ ID NOs: 113, 116, 118, 120, 121, 124, 126, and 128.

5. A method of administering to a cell a nucleic molecule of any of claims 1-4 comprising contacting said cell with the nucleic acid molecule under conditions suitable for said administration.

6. The method of claim 5, further comprising administering one or more other therapeutic compounds under conditions suitable for said administration.

7. The method of claim 6, wherein said other therapeutic compound is type I interferon.

8. The method of claim 6, wherein said other therapeutic compound is 3TC® (Lamivudine).

9. The method of claim 6, wherein said other therapeutic compound and the nucleic acid molecule are administered simultaneously.

10. The method of claim 6, wherein said other therapeutic compound and nucleic acid molecule are administered separately.

11. The method of claim 7, wherein said type I interferon is chosen from interferon alpha, interferon beta, consensus interferon, polyethylene glycol interferon, polyethylene glycol interferon alpha 2a, polyethylene glycol interferon alpha 2b, and polyethylene glycol consensus interferon

12. The nucleic acid molecule of any of claims 1-4, wherein said nucleic acid molecule comprises a nucleic acid backbone modification.

13. The nucleic acid decoy molecule of any of claims 1-4, wherein said nucleic acid molecule comprises a nucleic acid sugar modification.

14. The nucleic acid decoy molecule of any of claims 1-4, wherein said nucleic acid molecule comprises a nucleic acid base modification.

15. The method of claim 5, wherein said cell is a mammalian cell.

16. The method of claim 6, wherein said cell is a mammalian cell.

17. The method of claim 15, wherein said cell is a human cell.

18. The method of claim 16, wherein said cell is a human cell.

19. The method of claim 15, wherein said administration is in the presence of a delivery reagent.

20. The method of claim 16, wherein said administration is in the presence of a delivery reagent.

21. The method of claim 19, wherein said delivery reagent is a lipid.

22. The method of claim 21, wherein said lipid is a cationic lipid or a phospholipid.

23. The method of claim 19, wherein said delivery reagent is a liposome.

24. The method of claim 20, wherein said delivery reagent is a lipid.

25. The method of claim 24, wherein said lipid is a cationic lipid or a phospholipid.

26. The method of claim 20, wherein said delivery reagent is a lipsome.

27. The nucleic acid molecule of claim 1, wherein said nucleic acid molecule is a decoy nucleic acid molecule.

28. The nucleic acid molecule of claim 1, wherein said nucleic acid molecule is an aptamer nucleic acid molecule.

29. The nucleic acid molecule of claim 3, wherein said Enhancer I sequence comprises a Hepatocyte Nuclear Factor 3 and/or Hepatocyte Nuclear Factor 4 binding sequence.


SEQUENCE LISTING (128 sequences)

SEQ ID NO: 1 - RNA, 4 nt, Artificial Sequence
  Description of Artificial Sequence: HBV Decoy Nucleic Acid
auuc

SEQ ID NO: 2 - RNA, 4 nt, Artificial Sequence
  Description of Artificial Sequence: HBV Decoy Nucleic Acid
cauu

SEQ ID NO: 3 - RNA, 4 nt, Artificial Sequence
  Description of Artificial Sequence: HBV Decoy Nucleic Acid
ucau

SEQ ID NO: 4 - RNA, 4 nt, Artificial Sequence
  Description of Artificial Sequence: HBV Decoy Nucleic Acid
uuca

SEQ ID NO: 5 - RNA, 5 nt, Artificial Sequence
  Description of Artificial Sequence: HBV Decoy Nucleic Acid
auuca

SEQ ID NO: 6 - RNA, 5 nt, Artificial Sequence
  Description of Artificial Sequence: HBV Decoy Nucleic Acid
cauuc

SEQ ID NO: 7 - RNA, 5 nt, Artificial Sequence
  Description of Artificial Sequence: HBV Decoy Nucleic Acid
ucauu

SEQ ID NO: 8 - RNA, 5 nt, Artificial Sequence
  Description of Artificial Sequence: HBV Decoy Nucleic Acid
uucau

SEQ ID NO: 9 - RNA, 6 nt, Artificial Sequence
  Description of Artificial Sequence: HBV Decoy Nucleic Acid
auucau

SEQ ID NO: 10 - RNA, 6 nt, Artificial Sequence
  Description of Artificial Sequence: HBV Decoy Nucleic Acid
cauuca

SEQ ID NO: 11 - RNA, 6 nt, Artificial Sequence
  Description of Artificial Sequence: HBV Decoy Nucleic Acid
ucauuc

SEQ ID NO: 12 - RNA, 6 nt, Artificial Sequence
  Description of Artificial Sequence: HBV Decoy Nucleic Acid
uucauu

SEQ ID NO: 13 - RNA, 7 nt, Artificial Sequence
  Description of Artificial Sequence: HBV Decoy Nucleic Acid
auucauu

SEQ ID NO: 14 - RNA, 7 nt, Artificial Sequence
  Description of Artificial Sequence: HBV Decoy Nucleic Acid
cauucau

SEQ ID NO: 15 - RNA, 7 nt, Artificial Sequence
  Description of Artificial Sequence: HBV Decoy Nucleic Acid
ucauuca

SEQ ID NO: 16 - RNA, 7 nt, Artificial Sequence
  Description of Artificial Sequence: HBV Decoy Nucleic Acid
uucauuc

SEQ ID NO: 17 - RNA, 8 nt, Artificial Sequence
  Description of Artificial Sequence: HBV Decoy Nucleic Acid
auucauuc

SEQ ID NO: 18 - RNA, 8 nt, Artificial Sequence
  Description of Artificial Sequence: HBV Decoy Nucleic Acid
cauucauu

SEQ ID NO: 19 - RNA, 8 nt, Artificial Sequence
  Description of Artificial Sequence: HBV Decoy Nucleic Acid
ucauucau

SEQ ID NO: 20 - RNA, 8 nt, Artificial Sequence
  Description of Artificial Sequence: HBV Decoy Nucleic Acid
uucauuca

SEQ ID NO: 21 - RNA, 9 nt, Artificial Sequence
  Description of Artificial Sequence: HBV Decoy Nucleic Acid
auucauuca

SEQ ID NO: 22 - RNA, 9 nt, Artificial Sequence
  Description of Artificial Sequence: HBV Decoy Nucleic Acid
cauucauuc

SEQ ID NO: 23 - RNA, 9 nt, Artificial Sequence
  Description of Artificial Sequence: HBV Decoy Nucleic Acid
ucauucauu

SEQ ID NO: 24 - RNA, 9 nt, Artificial Sequence
  Description of Artificial Sequence: HBV Decoy Nucleic Acid
uucauucau

SEQ ID NO: 25 - RNA, 10 nt, Artificial Sequence
  Description of Artificial Sequence: HBV Decoy Nucleic Acid
auucauucau

SEQ ID NO: 26 - RNA, 10 nt, Artificial Sequence
  Description of Artificial Sequence: HBV Decoy Nucleic Acid
cauucauuca

SEQ ID NO: 27 - RNA, 10 nt, Artificial Sequence
  Description of Artificial Sequence: HBV Decoy Nucleic Acid
ucauucauuc

SEQ ID NO: 28 - RNA, 10 nt, Artificial Sequence
  Description of Artificial Sequence: HBV Decoy Nucleic Acid
uucauucauu

SEQ ID NO: 29 - RNA, 11 nt, Artificial Sequence
  Description of Artificial Sequence: HBV Decoy Nucleic Acid
auucauucauu

SEQ ID NO: 30 - RNA, 11 nt, Artificial Sequence
  Description of Artificial Sequence: HBV Decoy Nucleic Acid
cauucauucau

SEQ ID NO: 31 - RNA, 11 nt, Artificial Sequence
  Description of Artificial Sequence: HBV Decoy Nucleic Acid
ucauucauuca

SEQ ID NO: 32 - RNA, 11 nt, Artificial Sequence
  Description of Artificial Sequence: HBV Decoy Nucleic Acid
uucauucauuc

SEQ ID NO: 33 - RNA, 12 nt, Artificial Sequence
  Description of Artificial Sequence: HBV Decoy Nucleic Acid
auucauucauuc

SEQ ID NO: 34 - RNA, 12 nt, Artificial Sequence
  Description of Artificial Sequence: HBV Decoy Nucleic Acid
cauucauucauu

SEQ ID NO: 35 - RNA, 12 nt, Artificial Sequence
  Description of Artificial Sequence: HBV Decoy Nucleic Acid
ucauucauucau

SEQ ID NO: 36 - RNA, 12 nt, Artificial Sequence
  Description of Artificial Sequence: HBV Decoy Nucleic Acid
uucauucauuca

SEQ ID NO: 37 - RNA, 13 nt, Artificial Sequence
  Description of Artificial Sequence: HBV Decoy Nucleic Acid
auucauucauuca

SEQ ID NO: 38 - RNA, 13 nt, Artificial Sequence
  Description of Artificial Sequence: HBV Decoy Nucleic Acid
cauucauucauuc

SEQ ID NO: 39 - RNA, 13 nt, Artificial Sequence
  Description of Artificial Sequence: HBV Decoy Nucleic Acid
ucauucauucauu

SEQ ID NO: 40 - RNA, 13 nt, Artificial Sequence
  Description of Artificial Sequence: HBV Decoy Nucleic Acid
uucauucauucau

SEQ ID NO: 41 - RNA, 14 nt, Artificial Sequence
  Description of Artificial Sequence: HBV Decoy Nucleic Acid
auucauucauucau

SEQ ID NO: 42 - RNA, 14 nt, Artificial Sequence
  Description of Artificial Sequence: HBV Decoy Nucleic Acid
cauucauucauuca

SEQ ID NO: 43 - RNA, 14 nt, Artificial Sequence
  Description of Artificial Sequence: HBV Decoy Nucleic Acid
ucauucauucauuc

SEQ ID NO: 44 - RNA, 14 nt, Artificial Sequence
  Description of Artificial Sequence: HBV Decoy Nucleic Acid
uucauucauucauu

SEQ ID NO: 45 - RNA, 15 nt, Artificial Sequence
  Description of Artificial Sequence: HBV Decoy Nucleic Acid
auucauucauucauu

SEQ ID NO: 46 - RNA, 15 nt, Artificial Sequence
  Description of Artificial Sequence: HBV Decoy Nucleic Acid
cauucauucauucau

SEQ ID NO: 47 - RNA, 15 nt, Artificial Sequence
  Description of Artificial Sequence: HBV Decoy Nucleic Acid
ucauucauucauuca

SEQ ID NO: 48 - RNA, 15 nt, Artificial Sequence
  Description of Artificial Sequence: HBV Decoy Nucleic Acid
uucauucauucauuc

SEQ ID NO: 49 - RNA, 16 nt, Artificial Sequence
  Description of Artificial Sequence: HBV Decoy Nucleic Acid
auucauucauucauuc

SEQ ID NO: 50 - RNA, 16 nt, Artificial Sequence
  Description of Artificial Sequence: HBV Decoy Nucleic Acid
cauucauucauucauu

SEQ ID NO: 51 - RNA, 16 nt, Artificial Sequence
  Description of Artificial Sequence: HBV Decoy Nucleic Acid
ucauucauucauucau

SEQ ID NO: 52 - RNA, 16 nt, Artificial Sequence
  Description of Artificial Sequence: HBV Decoy Nucleic Acid
uucauucauucauuca

SEQ ID NO: 53 - RNA, 17 nt, Artificial Sequence
  Description of Artificial Sequence: HBV Decoy Nucleic Acid
auucauucauucauuca

SEQ ID NO: 54 - RNA, 17 nt, Artificial Sequence
  Description of Artificial Sequence: HBV Decoy Nucleic Acid
cauucauucauucauuc

SEQ ID NO: 55 - RNA, 17 nt, Artificial Sequence
  Description of Artificial Sequence: HBV Decoy Nucleic Acid
ucauucauucauucauu

SEQ ID NO: 56 - RNA, 17 nt, Artificial Sequence
  Description of Artificial Sequence: HBV Decoy Nucleic Acid
uucauucauucauucau

SEQ ID NO: 57 - RNA, 18 nt, Artificial Sequence
  Description of Artificial Sequence: HBV Decoy Nucleic Acid
auucauucauucauucau

SEQ ID NO: 58 - RNA, 18 nt, Artificial Sequence
  Description of Artificial Sequence: HBV Decoy Nucleic Acid
cauucauucauucauuca

SEQ ID NO: 59 - RNA, 18 nt, Artificial Sequence
  Description of Artificial Sequence: HBV Decoy Nucleic Acid
ucauucauucauucauuc

SEQ ID NO: 60 - RNA, 18 nt, Artificial Sequence
  Description of Artificial Sequence: HBV Decoy Nucleic Acid
uucauucauucauucauu

SEQ ID NO: 61 - RNA, 19 nt, Artificial Sequence
  Description of Artificial Sequence: HBV Decoy Nucleic Acid
auucauucauucauucauu

SEQ ID NO: 62 - RNA, 19 nt, Artificial Sequence
  Description of Artificial Sequence: HBV Decoy Nucleic Acid
cauucauucauucauucau

SEQ ID NO: 63 - RNA, 19 nt, Artificial Sequence
  Description of Artificial Sequence: HBV Decoy Nucleic Acid
ucauucauucauucauuca

SEQ ID NO: 64 - RNA, 19 nt, Artificial Sequence
  Description of Artificial Sequence: HBV Decoy Nucleic Acid
uucauucauucauucauuc

SEQ ID NO: 65 - RNA, 20 nt, Artificial Sequence
  Description of Artificial Sequence: HBV Decoy Nucleic Acid
auucauucauucauucauuc

SEQ ID NO: 66 - RNA, 20 nt, Artificial Sequence
  Description of Artificial Sequence: HBV Decoy Nucleic Acid
cauucauucauucauucauu

SEQ ID NO: 67 - RNA, 20 nt, Artificial Sequence
  Description of Artificial Sequence: HBV Decoy Nucleic Acid
ucauucauucauucauucau

SEQ ID NO: 68 - RNA, 20 nt, Artificial Sequence
  Description of Artificial Sequence: HBV Decoy Nucleic Acid
uucauucauucauucauuca

SEQ ID NO: 69 - RNA, 21 nt, Artificial Sequence
  Description of Artificial Sequence: HBV Decoy Nucleic Acid
auucauucauucauucauuca

SEQ ID NO: 70 - RNA, 21 nt, Artificial Sequence
  Description of Artificial Sequence: HBV Decoy Nucleic Acid
cauucauucauucauucauuc

SEQ ID NO: 71 - RNA, 21 nt, Artificial Sequence
  Description of Artificial Sequence: HBV Decoy Nucleic Acid
ucauucauucauucauucauu

SEQ ID NO: 72 - RNA, 21 nt, Artificial Sequence
  Description of Artificial Sequence: HBV Decoy Nucleic Acid
uucauucauucauucauucau

SEQ ID NO: 73 - RNA, 22 nt, Artificial Sequence
  Description of Artificial Sequence: HBV Decoy Nucleic Acid
cauucauucauucauucauuca

SEQ ID NO: 74 - RNA, 22 nt, Artificial Sequence
  Description of Artificial Sequence: HBV Decoy Nucleic Acid
ucauucauucauucauucauuc

SEQ ID NO: 75 - RNA, 22 nt, Artificial Sequence
  Description of Artificial Sequence: HBV Decoy Nucleic Acid
uucauucauucauucauucauu

SEQ ID NO: 76 - RNA, 23 nt, Artificial Sequence
  Description of Artificial Sequence: HBV Decoy Nucleic Acid
ucauucauucauucauucauuca

SEQ ID NO: 77 - RNA, 23 nt, Artificial Sequence
  Description of Artificial Sequence: HBV Decoy Nucleic Acid
uucauucauucauucauucauuc

SEQ ID NO: 78 - RNA, 24 nt, Artificial Sequence
  Description of Artificial Sequence: HBV Decoy Nucleic Acid
uucauucauucauucauucauuca

SEQ ID NO: 79 - RNA, 12 nt, Artificial Sequence
  Description of Artificial Sequence: HBV Decoy Nucleic Acid
  misc_feature (1)..(11): Phosphorothioate 3′-Internucleotide Linkage
  misc_feature (1)..(11): 2′-O-Allyl
  misc_feature (12)..(12): n stands for inverted deoxyabasic derivative
gcagaggugaan

SEQ ID NO: 80 - RNA, 12 nt, Artificial Sequence
  Description of Artificial Sequence: HBV Decoy Nucleic Acid
  misc_feature (1)..(11): Phosphorothioate 3′-Internucleotide Linkage
  misc_feature (1)..(11): 2′-O-Allyl
  misc_feature (12)..(12): n stands for inverted deoxyabasic derivative
aaguggagacgn

SEQ ID NO: 81 - RNA, 5 nt, Artificial Sequence
  Description of Artificial Sequence: HBV Decoy Nucleic Acid
  misc_feature (1)..(4): Phosphorothioate 3′-Internucleotide Linkage
  misc_feature (1)..(4): 2′-O-Allyl
  misc_feature (5)..(5): n stands for inverted deoxyabasic derivative
uucan

SEQ ID NO: 82 - RNA, 5 nt, Artificial Sequence
  Description of Artificial Sequence: HBV Decoy Nucleic Acid
  misc_feature (1)..(4): Phosphorothioate 3′-Internucleotide Linkage
  misc_feature (1)..(4): 2′-O-Allyl
  misc_feature (5)..(5): n stands for inverted deoxyabasic derivative
acuun

SEQ ID NO: 83 - RNA, 9 nt, Artificial Sequence
  Description of Artificial Sequence: HBV Decoy Nucleic Acid
  misc_feature (1)..(8): Phosphorothioate 3′-Internucleotide Linkage
  misc_feature (1)..(8): 2′-O-Allyl
  misc_feature (9)..(9): n stands for inverted deoxyabasic derivative
uucauucan

SEQ ID NO: 84 - RNA, 9 nt, Artificial Sequence
  Description of Artificial Sequence: HBV Decoy Nucleic Acid
  misc_feature (1)..(8): Phosphorothioate 3′-Internucleotide Linkage
  misc_feature (1)..(8): 2′-O-Allyl
  misc_feature (9)..(9): n stands for inverted deoxyabasic derivative
acuuacuun

SEQ ID NO: 85 - RNA, 13 nt, Artificial Sequence
  Description of Artificial Sequence: HBV Decoy Nucleic Acid
  misc_feature (1)..(12): Phosphorothioate 3′-Internucleotide Linkage
  misc_feature (1)..(12): 2′-O-Allyl
  misc_feature (13)..(13): n stands for inverted deoxyabasic derivative
uucauucauucan

SEQ ID NO: 86 - RNA, 13 nt, Artificial Sequence
  Description of Artificial Sequence: HBV Decoy Nucleic Acid
  misc_feature (1)..(12): Phosphorothioate 3′-Internucleotide Linkage
  misc_feature (1)..(12): 2′-O-Allyl
  misc_feature (13)..(13): n stands for inverted deoxyabasic derivative
acuuacuuacuun

SEQ ID NO: 87 - RNA, 17 nt, Artificial Sequence
  Description of Artificial Sequence: HBV Decoy Nucleic Acid
  misc_feature (1)..(16): Phosphorothioate 3′-Internucleotide Linkage
  misc_feature (1)..(16): 2′-O-Allyl
  misc_feature (17)..(17): n stands for inverted deoxyabasic derivative
uucauucauucauucan

SEQ ID NO: 88 - RNA, 17 nt, Artificial Sequence
  Description of Artificial Sequence: HBV Decoy Nucleic Acid
  misc_feature (1)..(16): Phosphorothioate 3′-Internucleotide Linkage
  misc_feature (1)..(16): 2′-O-Allyl
  misc_feature (17)..(17): n stands for inverted deoxyabasic derivative
acuuacuuacuuacuun

SEQ ID NO: 89 - RNA, 5 nt, Artificial Sequence
  Description of Artificial Sequence: HBV Decoy Nucleic Acid
  misc_feature (1)..(4): Phosphorothioate 3′-Internucleotide Linkage
  misc_feature (1)..(4): 2′-O-Allyl
  misc_feature (5)..(5): n stands for inverted deoxyabasic derivative
ugaan

SEQ ID NO: 90 - RNA, 5 nt, Artificial Sequence
  Description of Artificial Sequence: HBV Decoy Nucleic Acid
  misc_feature (1)..(4): Phosphorothioate 3′-Internucleotide Linkage
  misc_feature (1)..(4): 2′-O-Allyl
  misc_feature (5)..(5): n stands for inverted deoxyabasic derivative
aagun

SEQ ID NO: 91 - RNA, 8 nt, Artificial Sequence
  Description of Artificial Sequence: HBV Decoy Nucleic Acid
  misc_feature (1)..(7): Phosphorothioate 3′-Internucleotide Linkage
  misc_feature (1)..(7): 2′-O-Allyl
  misc_feature (8)..(8): n stands for inverted deoxyabasic derivative
gcuugaan

SEQ ID NO: 92 - RNA, 8 nt, Artificial Sequence
  Description of Artificial Sequence: HBV Decoy Nucleic Acid
  misc_feature (1)..(7): Phosphorothioate 3′-Internucleotide Linkage
  misc_feature (1)..(7): 2′-O-Allyl
  misc_feature (8)..(8): n stands for inverted deoxyabasic derivative
aaguucgn

SEQ ID NO: 93 - RNA, 12 nt, Artificial Sequence
  Description of Artificial Sequence: HBV Decoy Nucleic Acid
  misc_feature (1)..(10): Phosphorothioate 3′-Internucleotide Linkage
  misc_feature (1)..(11): 2′-O-Allyl
  misc_feature (12)..(12): n stands for inverted deoxyabasic derivative
ggaggcuugaan

SEQ ID NO: 94 - RNA, 12 nt, Artificial Sequence
  Description of Artificial Sequence: HBV Decoy Nucleic Acid
  misc_feature (1)..(11): Phosphorothioate 3′-Internucleotide Linkage
  misc_feature (1)..(11): 2′-O-Allyl
  misc_feature (12)..(12): n stands for inverted deoxyabasic derivative
aaguucggaggn

SEQ ID NO: 95 - RNA, 37 nt, Artificial Sequence
  Description of Artificial Sequence: Enzymatic Nucleic Acid
  misc_feature (1)..(4): Phosphorothioate 3′-Internucleotide Linkage
  misc_feature (1)..(8): 2′-O-Methyl
  misc_feature (12)..(12): 2′-O-Methyl
  misc_feature (14)..(26): 2′-O-Methyl
  misc_feature (28)..(29): 2′-O-Methyl
  misc_feature (31)..(36): 2′-O-Methyl
  misc_feature (9)..(9): 2′-deoxy-2′-C-Allyl
  misc_feature (37)..(37): n stands for inverted deoxyabasic derivative
gaaaauucugaugaggccguuaggccgaaagagaagn

SEQ ID NO: 96 - RNA, 37 nt, Artificial Sequence
  Description of Artificial Sequence: Enzymatic Nucleic Acid
  misc_feature (1)..(4): Phosphorothioate 3′-Internucleotide Linkage
  misc_feature (1)..(8): 2′-O-Methyl
  misc_feature (12)..(12): 2′-O-Methyl
  misc_feature (14)..(26): 2′-O-Methyl
  misc_feature (28)..(29): 2′-O-Methyl
  misc_feature (31)..(36): 2′-O-Methyl
  misc_feature (9)..(9): 2′-deoxy-2′-C-Allyl
  misc_feature (37)..(37): n stands for inverted deoxyabasic derivative
aaugaggcuagugacgccguuaggcggaaaaaugaan

SEQ ID NO: 97 - RNA, 5 nt, Artificial Sequence
  Description of Artificial Sequence: HBV Decoy Nucleic Acid
  misc_feature (1)..(4): 2′-O-Methyl
  misc_feature (5)..(5): n stands for inverted deoxyabasic derivative
ugaan

SEQ ID NO: 98 - RNA, 5 nt, Artificial Sequence
  Description of Artificial Sequence: HBV Decoy Nucleic Acid
  misc_feature (1)..(4): 2′-O-Methyl
  misc_feature (5)..(5): n stands for inverted deoxyabasic derivative
aagun

SEQ ID NO: 99 - RNA, 12 nt, Artificial Sequence
  Description of Artificial Sequence: HBV Decoy Nucleic Acid
  misc_feature (1)..(11): 2′-O-Methyl
  misc_feature (12)..(12): n stands for inverted deoxyabasic derivative
ggaggcuugaan

SEQ ID NO: 100 - RNA, 12 nt, Artificial Sequence
  Description of Artificial Sequence: HBV Decoy Nucleic Acid
  misc_feature (1)..(11): 2′-O-Methyl
  misc_feature (12)..(12): n stands for inverted deoxyabasic derivative
aaguucggaggn

SEQ ID NO: 101 - RNA, 8 nt, Artificial Sequence
  Description of Artificial Sequence: HBV Decoy Nucleic Acid
  misc_feature (1)..(7): 2′-O-Methyl
  misc_feature (8)..(8): n stands for inverted deoxyabasic derivative
gcuugaan

SEQ ID NO: 102 - RNA, 8 nt, Artificial Sequence
  Description of Artificial Sequence: HBV Decoy Nucleic Acid
  misc_feature (1)..(7): 2′-O-Methyl
  misc_feature (8)..(8): n stands for inverted deoxyabasic derivative
aaguucgn

SEQ ID NO: 103 - RNA, 12 nt, Artificial Sequence
  Description of Artificial Sequence: HBV Decoy Nucleic Acid
  misc_feature (1)..(11): 2′-O-Methyl
  misc_feature (12)..(12): n stands for inverted deoxyabasic derivative
gcagaggugaan

SEQ ID NO: 104 - RNA, 12 nt, Artificial Sequence
  Description of Artificial Sequence: HBV Decoy Nucleic Acid
  misc_feature (1)..(11): 2′-O-Methyl
  misc_feature (12)..(12): n stands for inverted deoxyabasic derivative
aaguggagacgn

SEQ ID NO: 105 - RNA, 5 nt, Artificial Sequence
  Description of Artificial Sequence: HBV Decoy Nucleic Acid
  misc_feature (1)..(4): 2′-O-Methyl
  misc_feature (5)..(5): n stands for inverted deoxyabasic derivative
uucan

SEQ ID NO: 106 - RNA, 5 nt, Artificial Sequence
  Description of Artificial Sequence: HBV Decoy Nucleic Acid
  misc_feature (1)..(4): 2′-O-Methyl
  misc_feature (5)..(5): n stands for inverted deoxyabasic derivative
acuun

SEQ ID NO: 107 - RNA, 9 nt, Artificial Sequence
  Description of Artificial Sequence: HBV Decoy Nucleic Acid
  misc_feature (1)..(8): 2′-O-Methyl
  misc_feature (9)..(9): n stands for inverted deoxyabasic derivative
uucauucan

SEQ ID NO: 108 - RNA, 9 nt, Artificial Sequence
  Description of Artificial Sequence: HBV Decoy Nucleic Acid
  misc_feature (1)..(8): 2′-O-Methyl
  misc_feature (9)..(9): n stands for inverted deoxyabasic derivative
acuuacuun

SEQ ID NO: 109 - RNA, 13 nt, Artificial Sequence
  Description of Artificial Sequence: HBV Decoy Nucleic Acid
  misc_feature (1)..(12): 2′-O-Methyl
  misc_feature (13)..(13): n stands for inverted deoxyabasic derivative
uucauucauucan

SEQ ID NO: 110 - RNA, 13 nt, Artificial Sequence
  Description of Artificial Sequence: HBV Decoy Nucleic Acid
  misc_feature (1)..(12): 2′-O-Methyl
  misc_feature (13)..(13): n stands for inverted deoxyabasic derivative
acuuacuuacuun

SEQ ID NO: 111 - RNA, 17 nt, Artificial Sequence
  Description of Artificial Sequence: HBV Decoy Nucleic Acid
  misc_feature (1)..(16): 2′-O-Methyl
  misc_feature (17)..(17): n stands for inverted deoxyabasic derivative
uucauucauucauucan

SEQ ID NO: 112 - RNA, 17 nt, Artificial Sequence
  Description of Artificial Sequence: HBV Decoy Nucleic Acid
  misc_feature (1)..(16): 2′-O-Methyl
  misc_feature (17)..(17): n stands for inverted deoxyabasic derivative
acuuacuuacuuacuun

SEQ ID NO: 113 - RNA, 18 nt, Artificial Sequence
  Description of Artificial Sequence: HBV DNA Binding Nucleic Acid
  misc_feature (1)..(1): n stands for inverted deoxyabasic derivative
  misc_feature (18)..(18): n stands for inverted deoxyabasic derivative
  misc_feature (2)..(16): Phosphorothioate 3′-Internucleotide Linkage
  misc_feature (2)..(17): 2′-O-Methyl
nuuucuaaguaaacagun

SEQ ID NO: 114 - RNA, 18 nt, Artificial Sequence
  Description of Artificial Sequence: HBV DNA Binding Nucleic Acid
  misc_feature (1)..(1): n stands for inverted deoxyabasic derivative
  misc_feature (18)..(18): n stands for inverted deoxyabasic derivative
  misc_feature (2)..(16): Phosphorothioate 3′-Internucleotide Linkage
  misc_feature (2)..(17): 2′-O-Methyl
nacuguuuacuuagaaan

SEQ ID NO: 115 - RNA, 18 nt, Artificial Sequence
  Description of Artificial Sequence: HBV DNA Binding Nucleic Acid
  misc_feature (1)..(1): n stands for inverted deoxyabasic derivative
  misc_feature (18)..(18): n stands for inverted deoxyabasic derivative
  misc_feature (2)..(16): Phosphorothioate 3′-Internucleotide Linkage
  misc_feature (2)..(17): 2′-O-Methyl
naaguaacucuauguuan

SEQ ID NO: 116 - RNA, 20 nt, Artificial Sequence
  Description of Artificial Sequence: HBV DNA Binding Nucleic Acid
  misc_feature (1)..(1): n stands for inverted deoxyabasic derivative
  misc_feature (20)..(20): n stands for inverted deoxyabasic derivative
  misc_feature (2)..(18): Phosphorothioate 3′-Internucleotide Linkage
  misc_feature (2)..(19): 2′-O-Methyl
nuacaugaaccuuuaccccn

SEQ ID NO: 117 - RNA, 19 nt, Artificial Sequence
  Description of Artificial Sequence: HBV DNA Binding Nucleic Acid
  misc_feature (1)..(1): n stands for inverted deoxyabasic derivative
  misc_feature (19)..(19): n stands for inverted deoxyabasic derivative
  misc_feature (2)..(17): Phosphorothioate 3′-Internucleotide Linkage
  misc_feature (2)..(18): 2′-O-Methyl
nggguaaagguucauguan

SEQ ID NO: 118 - RNA, 20 nt, Artificial Sequence
  Description of Artificial Sequence: HBV DNA Binding Nucleic Acid
  misc_feature (1)..(1): n stands for inverted deoxyabasic derivative
  misc_feature (20)..(20): n stands for inverted deoxyabasic derivative
  misc_feature (2)..(18): Phosphorothioate 3′-Internucleotide Linkage
  misc_feature (2)..(19): 2′-O-Methyl
naccuaucgccuacucuaan

SEQ ID NO: 119 - RNA, 19 nt, Artificial Sequence
  Description of Artificial Sequence: HBV DNA Binding Nucleic Acid
  misc_feature (1)..(1): n stands for inverted deoxyabasic derivative
  misc_feature (19)..(19): n stands for inverted deoxyabasic derivative
  misc_feature (2)..(17): Phosphorothioate 3′-Internucleotide Linkage
  misc_feature (2)..(18): 2′-O-Methyl
nugauagcggaugagauun

SEQ ID NO: 120 - RNA, 13 nt, Artificial Sequence
  Description of Artificial Sequence: HBV DNA Binding Nucleic Acid
  misc_feature (1)..(1): n stands for inverted deoxyabasic derivative
  misc_feature (13)..(13): n stands for inverted deoxyabasic derivative
  misc_feature (2)..(11): Phosphorothioate 3′-Internucleotide Linkage
  misc_feature (2)..(12): 2′-O-Methyl
nuucaccucugcn

SEQ ID NO: 121 - RNA, 18 nt, Artificial Sequence
  Description of Artificial Sequence: HBV DNA Binding Nucleic Acid
  misc_feature (1)..(1): n stands for inverted deoxyabasic derivative
  misc_feature (18)..(18): n stands for inverted deoxyabasic derivative
  misc_feature (2)..(16): Phosphorothioate 3′-Internucleotide Linkage
  misc_feature (2)..(17): 2′-O-Allyl
nuuucuaaguaaacagun

SEQ ID NO: 122 - RNA, 18 nt, Artificial Sequence
  Description of Artificial Sequence: HBV DNA Binding Nucleic Acid
  misc_feature (1)..(1): n stands for inverted deoxyabasic derivative
  misc_feature (18)..(18): n stands for inverted deoxyabasic derivative
  misc_feature (2)..(16): Phosphorothioate 3′-Internucleotide Linkage
  misc_feature (2)..(17): 2′-O-Allyl
nacuguuuacuuagaaan

SEQ ID NO: 123 - RNA, 18 nt, Artificial Sequence
  Description of Artificial Sequence: HBV DNA Binding Nucleic Acid
  misc_feature (1)..(1): n stands for inverted deoxyabasic derivative
  misc_feature (18)..(18): n stands for inverted deoxyabasic derivative
  misc_feature (2)..(16): Phosphorothioate 3′-Internucleotide Linkage
  misc_feature (2)..(17): 2′-O-Allyl
naaguaacucuauguuan

SEQ ID NO: 124 - RNA, 20 nt, Artificial Sequence
  Description of Artificial Sequence: HBV DNA Binding Nucleic Acid
  misc_feature (1)..(1): n stands for inverted deoxyabasic derivative
  misc_feature (20)..(20): n stands for inverted deoxyabasic derivative
  misc_feature (2)..(18): Phosphorothioate 3′-Internucleotide Linkage
  misc_feature (2)..(19): 2′-O-Allyl
nuacaugaaccuuuaccccn

SEQ ID NO: 125 - RNA, 19 nt, Artificial Sequence
  Description of Artificial Sequence: HBV DNA Binding Nucleic Acid
  misc_feature (1)..(1): n stands for inverted deoxyabasic derivative
  misc_feature (19)..(19): n stands for inverted deoxyabasic derivative
  misc_feature (2)..(17): Phosphorothioate 3′-Internucleotide Linkage
  misc_feature (2)..(18): 2′-O-Allyl
nggguaaagguucauguan

SEQ ID NO: 126 - RNA, 20 nt, Artificial Sequence
  Description of Artificial Sequence: HBV DNA Binding Nucleic Acid
  misc_feature (1)..(1): n stands for inverted deoxyabasic derivative
  misc_feature (20)..(20): n stands for inverted deoxyabasic derivative
  misc_feature (2)..(18): Phosphorothioate 3′-Internucleotide Linkage
  misc_feature (2)..(19): 2′-O-Allyl
naccuaucgccuacucuaan

SEQ ID NO: 127 - RNA, 19 nt, Artificial Sequence
  Description of Artificial Sequence: HBV DNA Binding Nucleic Acid
  misc_feature (1)..(1): n stands for inverted deoxyabasic derivative
  misc_feature (19)..(19): n stands for inverted deoxyabasic derivative
  misc_feature (2)..(17): Phosphorothioate 3′-Internucleotide Linkage
  misc_feature (2)..(18): 2′-O-Allyl
nugauagcggaugagauun

SEQ ID NO: 128 - RNA, 13 nt, Artificial Sequence
  Description of Artificial Sequence: HBV DNA Binding Nucleic Acid
  misc_feature (1)..(1): n stands for inverted deoxyabasic derivative
  misc_feature (13)..(13): n stands for inverted deoxyabasic derivative
  misc_feature (2)..(11): Phosphorothioate 3′-Internucleotide Linkage
  misc_feature (2)..(12): 2′-O-Allyl
nuucaccucugcn